(19) United States
(12) Patent Application Publication
(10) Pub. No.: US 2025/0320480 A1
(43) Pub. Date: Oct. 16, 2025

(54) Immunoglobulin Cleaving Enzyme

(71) Applicant: Genovis AB, Kaevlinge (SE)

(72) Inventors: Rolf Lood-Alayón, Kaevlinge (SE); Fredrik Olsson, Kaevlinge (SE)

(21) Appl. No.: 18/287,565

(22) PCT Filed: Apr. 22, 2022
(86) PCT No.: PCT/EP2022/060766

§ 371 Date: Oct. 19, 2023

(30) Foreign Application Priority Data
Apr. 22, 2021 (EP) 21169774.3

(51) Int. Cl. C12N 9/52; A61K 38/00; C12N 15/70

(52) U.S. Cl. CPC C12N 9/52; C12N 15/70; A61K 38/00

(57) ABSTRACT

The present invention relates to a novel polypeptide which displays protease activity against immunoglobulins, particularly human IgG, and in vivo, in vitro and ex vivo uses thereof. Uses of the polypeptide include methods for the analysis of IgG and the generation of antibody fragments, as well as methods for the prevention or treatment of diseases and conditions mediated by IgG.

### FIELD OF THE INVENTION

The present invention relates to a novel polypeptide which displays protease activity against immunoglobulins, particularly human IgG, and in vivo and ex vivo uses thereof. Uses of the polypeptide include methods for the analysis of IgG and the generation of antibody fragments, as well as methods for the prevention or treatment of diseases and conditions mediated by IgG.

### BACKGROUND OF THE INVENTION

IdeS (Immunoglobulin G-degrading enzyme of S. pyogenes, also known as imlifidase) is an extracellular cysteine protease produced by the human pathogen S. pyogenes. IdeS has an extraordinarily high degree of substrate specificity, with its only identified substrate being IgG. IdeS catalyses a single proteolytic cleavage in the lower hinge region of the heavy chains of all subclasses of human IgG. IdeS also catalyses an equivalent cleavage of the heavy chains of some subclasses of IgG in various animals. IdeS efficiently cleaves IgG to Fc and F(ab′)₂ fragments via a two-stage mechanism. In the first stage, one (first) heavy chain of IgG is cleaved to generate a single cleaved IgG (scIgG) molecule with a single non-covalently bound Fc chain. The scIgG molecule is effectively an intermediate product which retains the remaining (second) heavy chain of the original IgG molecule. In the second stage of the mechanism this second heavy chain is cleaved by IdeS to release a F(ab′)₂ fragment and a homodimeric Fc fragment. These are the products generally observed under physiological conditions. The homodimeric Fc may dissociate into its component monomers. Under reducing conditions the F(ab′)₂ fragment may dissociate to two Fab fragments. The IgG cleaving ability of IdeS has been shown to have utility ex vivo, for example in methods for production of Fab, F(ab′)₂ and Fc fragments, which may be used e.g. for the analysis of IgG and in vitro generation of F(ab′)₂ fragments. See, for example, WO2003051914 and WO2009033670. IdeS and IgG cleaving proteins developed by modification of IdeS have also been shown to have in vivo utility as therapeutic agents, see for example, WO2006131347, WO2013110946, WO2016012285, WO2016128558, and WO2016128559. Such proteins are useful in this context because they are capable of the in vivo cleavage of IgG molecules which mediate disease or which are otherwise undesirable. The removal of IgG can be beneficial because IgG contributes to the pathology of many autoimmune conditions as well as to acute rejection of transplanted organs, and may also contribute to anti-drug responses. Antibodies specific for a given therapeutic agent may reduce its efficacy. This is particularly the case for antibody-based therapeutics, gene therapy vectors, and cell therapies including adoptive cell transfer (ACT) immunotherapies e.g. using CAR-T cells. In other words, IdeS and IgG cleaving proteins developed by modification of IdeS may be used as a therapy for any disease or condition wholly or partly mediated by IgG, which condition may include anti-drug responses.

However, IdeS is a virulence factor of S. pyogenes, which is responsible for common infections like tonsillitis and strep throat. Accordingly, most human subjects have encountered IdeS in this context and are likely to have anti-IdeS antibodies in the bloodstream, which may reduce its utility as a therapeutic agent.

IdeZ is an IgG cysteine protease produced by Streptococcus equi ssp. zooepidemicus, a bacterium predominantly found in horses. IdeZ has approximately 66% identity to IdeS. Since Streptococcus equi ssp. zooepidemicus is not a human pathogen, IdeZ was considered to be an alternative to IdeS-based therapies because humans may have fewer or no antibodies (anti-drug antibodies, ADA) against IdeZ. However, IdeZ has a level of IgG cysteine protease activity against human IgG which is considerably lower than that of IdeS, in particular when cleaving IgG2.

Given the multiple applications for immunoglobulin cleaving enzymes outlined above, there is a clear need for additional agents which are capable of cleaving immunoglobulins, particularly human IgG.

### SUMMARY OF THE INVENTION

The present inventors have identified, purified and characterised a novel polypeptide. The polypeptide was identified in a previously unknown streptococcal species isolated as part of a study in multiple rodents. One particular isolated species, tentatively called Streptococcus krösus, was sequenced and was found to express several glycosidases and proteases. Surprisingly, a polypeptide was identified in an oral Streptococci. No IgG protease has to date been found in any of the oral Streptococci.

A polypeptide of approximately 56.4 kDa, consisting of 557 amino acids was identified and is referred to herein as IdeSORK. The full length sequence is shown in SEQ ID NO: 1. The sequence of IdeSORK is considerably different to that of other known IgG cysteine proteases, and IdeSORK clusters separately from IdeS and IdeZ in a phylogenetic analysis. Furthermore, the full length sequence is relatively difficult to express. However, the inventors have found that N terminal fragments of the full length sequence have significantly enhanced expression, whilst preserving immunoglobulin cleaving activity.

For example, the sequence of SEQ ID NO: 2 consists of the 312 amino acids from the N terminus of SEQ ID NO: 1. A polypeptide consisting of a version of the sequence of SEQ ID NO: 2 which is engineered for better expression and purification (addition of N terminal methionine and a C-terminal protein purification tag—His6) is referred to herein as IdeSORK2.0 (or Xork), the sequence of which is shown in SEQ ID NO: 3.

IdeSORK2.0 has immunoglobulin cleaving activity in the presence of human serum. Furthermore, there is very little pre-existing immunity against IdeSORK2.0. Thus, highly advantageously, the present invention allows the treatment of patients that are currently excluded from treatment due to pre-existing immunity to IdeS/IdeZ.

Provided herein is:

A polypeptide, optionally an engineered polypeptide, having immunoglobulin protease activity, wherein said polypeptide comprises, consists essentially of, or consists of:
(a) the amino acid sequence of SEQ ID NO: 1;
(b) the amino acid sequence of SEQ ID NO: 2;
(c) an amino acid sequence which is an N terminal fragment of the sequence of SEQ ID NO: 1;
(d) an amino acid sequence which is at least 50% identical to the amino acid sequence of any one of (a), (b) or (c);
optionally wherein said polypeptide is engineered to include an additional methionine at the N terminus and/or a protein purification or other tag at the C terminus, which tag may be joined to the C terminus by a linker.

The polypeptide may be provided in a composition, typically comprising an effective amount of a preservative. The polypeptide may be immobilised.

Also provided is a polynucleotide or expression vector which comprises a nucleic acid sequence encoding a polypeptide of the invention.

The polypeptides of the invention are useful in various methods for cleaving immunoglobulins, typically IgG. Thus, provided herein are methods comprising administering a polypeptide, polynucleotide, expression vector or composition of the invention, for example to a sample or a subject in which IgG is present. Also provided are methods comprising contacting IgG with a polypeptide, polynucleotide, expression vector or composition of the invention. The method may be a method of cleaving an immunoglobulin, and may optionally further comprise the detection or analysis of the cleavage products. The method may be a method treating a disease, the method comprising administering a polypeptide, polynucleotide, expression vector or composition of the invention to a subject.

### BRIEF DESCRIPTION OF THE FIGURES

FIG. 1: Expression and purification of IdeSork. The full length protein (1.0) is expressed as a ca 70 kDa protein, but expresses poorly. The C-terminally truncated IdeSork (2.0) expresses as a ca 36 kDa protein. Some material is insoluble (pellet) using the specified production protocol, but the vast majority can be solubilized (supernatant) and affinity-purified to high purity and yield.

FIG. 2. General protease activity. General protease activity was measured with the EnzCHECK protease assay, using casein as a substrate. The positive control FabULOUS is a general protease (SpeB) giving a high signal, while the negative control FabRICATOR is an IgG-specific protease (IdeS), thus giving almost no signal. IdeSork 2.0 did not give any measurable signal in the assay, indicating a more specific enzyme.

FIG. 3. Specificity of IdeSork2.0. IdeSork2.0 was mixed with different antibodies and incubated o/n at 37° C. before analysis on SDS-PAGE. The enzyme itself is noted with an arrow. All human IgG, or fusion proteins based on IgG, were hydrolyzed by IdeSork2.0, while only IgG2a from mouse could be hydrolyzed. Mouse IgG1, mouse IgG2b and human IgA were not affected by IdeSork2.0.

FIG. 4. Protease inhibitor sensitivity of IdeSork2.0. IdeSork2.0 is inhibited by most protease inhibitors, besides aprotinin, EDTA, and pepstatin. Bestatin is also only partly inhibitory to the enzyme.

FIG. 5. IdeSork2.0 hydrolyses the hinge of IgG at an identical site as IdeS. Commercial and non-commercial biologics were hydrolyzed with IdeSork2.0 and hydrolysis products were analyzed using LC-MS. In all cases hydrolysis was only observed at a single site in the hinge region, corresponding to the same site as IdeS recognizes.

FIG. 6. IdeSork2.0 is acting upon IgG in a two-step mechanism. To investigate if IdeSork2.0 is hydrolyzing both heavy chains of IgG simultaneously, or acting on them individually, we incubated Trastuzumab with IdeSork2.0 (1:20) and incubated for 0-60 minutes before analyzing on a non-reduced SDS-PAGE. Most IgG is hydrolyzed within the first few minutes, with one and two chains being hydrolyzed, indicating that it is a two-step reaction.

FIG. 7. IdeSork2.0 optimal conditions. The optimal buffer composition for IdeSork2.0 was determined using a Box-Behnken experimental design, measuring the impact of pH, NaCl and temperature. Output was measures as percentage hydrolysis of IgG.

FIG. 8. IdeSork2.0 enzyme: substrate ratio. The impact of enzyme and substrate concentration for the reaction speed was investigated using a Box-Behnken experimental setting, also analyzing the impact of time for the reaction speed. Output was measured as percentage hydrolysis of IgG (top left figure), as well as quantity hydrolyzed IgG per unit enzyme (all other figures).

FIG. 9. IdeSork2.0 enzyme: substrate activity over time. To verify the impact of modifying the enzyme: substrate ratio, an experiment with varying amounts of substrate (5-40 μg) to a set amount of enzyme (1 μg) was conducted over time (30, 60, and 120 min). Almost all IgG is hydrolyzed within 30 minutes at all conditions.

FIG. 10. IdeSork2.0 is having a similar activity compared to IdeS and IdeZ. To investigate if IdeSork2.0 is hydrolyzing IgG in a similar way as IdeS and IdeZ they were all incubated against IgG1 and IgG2 and analyzed on SDS-PAGE. The cleavage patterns look identical, with both IdeSork2.0 and IdeS having less activity towards IgG2.

FIG. 11. Specificity of IdeSork2.0. IdeSork2.0 was mixed with different antibodies and incubated o/n at 37° C. before analysis on SDS-PAGE. IdeSORK2.0 had no visible effect on sheep, pig, dog, or rat IgG, but showed activity on horse and guinea pig IgG.

FIG. 12. Identification of IgG protease activity in Streptococcal strains isolated from voles. (A) Eight beta-hemolytic bacteria isolated from wild voles were subjected to 16S gene amplification, using S. oralis as a positive control. (B) The sequences were queried against databases with known 16S sequences, demonstrating similarities with mainly oral streptococci and staphylococci. (C) To investigate IgG protease activity, supernatant from several strains grown in C-medium supplemented with 2% mouse serum were subjected to Western blot. Only strain β13 and β2003 showed measurable activity against mouse IgG and were sequenced in full. (D) Whole genome sequence homology demonstrate the evolutionary relationship between the newly isolated strains and oral streptococci. No IgG protease has to date been found in any of the oral streptococci or close relatives, and the gene for IdeSORK was only identified in one of the sequenced strains.

FIG. 13. (A) Clustal W alignment of the IgG proteases IdeSORK1.0, IdeSORK2.0, IdeS and IdeZ. (B) IdeSork2.0 was synthesized with a truncated C-terminal, and expressed and tested for yield and activity. “0” represents the final IdeSORK2.0 construct, and +/− describes how many amino acids in the C-terminus have been removed or added to that product.

FIG. 14. IgG hydrolase homology. (A) The three IgG-specific hydrolases IdeS, IdeZ, and IdeSORK2.0 (Xork) were separated on SDS-PAGE. (B) The homology of Xork to other known IgG-proteases was assessed through a phylogenetic tree. (C) Percentage similarity and identity of Xork to other known IgG-proteases. (D) Sequence alignments for Xork vs IdeS and (E) Xork vs IdeZ.

FIG. 15. Activity of IgG hydrolases in human serum. Serum (100%) was spiked with IgG hydrolases and activity was analyzed over time using non-reduced SDS-PAGE.

FIG. 16. Enzymatic activity towards IgG. The activity of IdeS, IdeZ, and Xork towards human IgG subclasses, modified IgG, fusion proteins with IgG1-Fc-parts, and IgG from different species is reported as not active (−), low activity (+), good activity (++), and great activity (+++). Relates to FIG. 3.

FIG. 17. Denatured IgG was incubated with IgG hydrolases to investigate the necessity of a conserved 3D structure of the substrate for the enzymes to exert their function.

FIG. 18. Prevalence of anti-IgG hydrolase antibodies in IVIG. For both an (A) ELISA assay and a (B) Western blot assay, IgG hydrolases at different concentrations were bound to a plate/membrane and exposed to IVIG (5 ug/mL). A secondary antibody, goat-anti IgG was added having a fused HRP-tag, allowing for signal amplification.

### BRIEF DESCRIPTION OF THE SEQUENCES

SEQ ID NO: 1 is the full length amino acid sequence of a polypeptide isolated from a streptococcal species designated Streptococcus krösus. The polypeptide is 557 amino acids in length and has immunoglobulin protease activity. It may be referred to herein as IdeSORK.

SEQ ID NO: 2 is an amino acid sequence of a polypeptide having immunoglobulin protease activity. The C-terminal of 245 amino acids of SEQ ID NO: 1 are deleted to form this sequence, which thus consists of the N terminal 312 amino acids of IdeSORK. This sequence may be described as a N terminal fragment of IdeSORK having immunoglobulin protease activity.

SEQ ID NO: 3 is an amino acid sequence of a polypeptide having immunoglobulin protease activity. This sequence consists of the sequence of SEQ ID NO: 2, engineered to include an additional N terminal methionine and a C-terminal protein purification tag-His6. The resulting 319 amino acid polypeptide may be referred to herein as IdeSORK2.0.

SEQ ID NOs: 4 to 11 are the sequences of the Hinge/CH2 regions of various human and mouse IgG subclasses.

SEQ ID NO: 12 is the full length amino acid sequence of IdeS, which is publically available as NCBI Reference Sequence no. WP_010922160.1. This sequence includes an N terminal methionine followed by a 28 amino acid secretion signal sequence.

SEQ ID NO: 13 is the mature IdeS protein. The N terminal methionine and the signal sequence (a total of 29 amino acids at the N terminus) are removed from SEQ ID NO: 12 to form this sequence, which is also publically available as Genbank accession no. ADF13949.1. When used in the Examples, IdeS typically comprises this sequence engineered to include an additional N terminal methionine and a C-terminal protein purification tag, such as poly-histidine. A preferred tag is His6.

SEQ ID NO: 14 is the full length amino acid sequence of IdeZ, which is publically available as NCBI Reference Sequence no WP_014622780.1. This sequence includes an N terminal methionine followed by a 33 amino acid secretion signal sequence.

SEQ ID NO: 15 is the mature IdeZ protein. The N terminal methionine and the signal sequence (a total of 34 amino acids at the N terminus) are removed from SEQ ID NO: 14 to form this sequence. When used in the Examples, IdeZ typically comprises this sequence engineered to include an additional N terminal methionine and a C-terminal protein purification tag, such as poly-histidine. A preferred tag is His6.

SEQ ID NO: 16 is an exemplary nucleotide sequence encoding the polypeptide of SEQ ID NO: 1 engineered for expression with an N terminal histidine and a C terminal His6.

SEQ ID NO: 17 is an exemplary nucleotide sequence encoding the polypeptide of SEQ ID NO: 2 engineered for expression with an N terminal histidine and a C terminal His6. SEQ ID NO: 17 encodes SEQ ID NO: 3.

SEQ ID NO: 18 is an exemplary expression vector sequence comprising the sequence of SEQ ID NO: 16.

SEQ ID NO: 19 is an exemplary expression vector sequence comprising the sequence of SEQ ID NO: 17.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting. All publications, patents and patent applications cited herein, whether supra or infra, are hereby incorporated by reference in their entirety.

As used in this specification and the appended claims, the singular forms “a”, “an”, and “the” include plural referents unless the content clearly dictates otherwise. Thus, for example, reference to “a polypeptide” includes “polypeptides”, and the like.

### Polypeptides Having Immunoglobulin Protease Activity

### Functional Features of a Polypeptide Having Immunoglobulin Protease Activity

This section sets out the functional features of a polypeptide having immunoglobulin protease activity, which may apply in addition to the structural features outlined in the immediately following section.

The present invention provides a polypeptide having immunoglobulin protease activity. That is, the polypeptide is able to cleave an immunoglobulin molecule. The immunoglobulin molecule is typically an IgG molecule, and preferably the polypeptide does not cleave other classes of immunoglobulin, The polypeptide may cleave any immunoglobulin molecule comprising a hinge/CH2 sequence of any one of SEQ ID NOs. 4 to 8 as shown in Table 1.

TABLE 1

| Subclass | Hinge/CH2 | SEQ | Subclass | Hinge/CH2 | SEQ |
|---|---|---|---|---|---|
| Human | sequence | ID | Mouse | sequence | ID |
| IgG1 | CPPCPAPELLGGPSVF | 4 | IgG1 | PCICTVPEVSSVF | 10 |
| IgG2 | CPPCPAPPVAGPSVF | 5 | IgG2a | CPPCAAPNLLGGPSVF | 8 |
| IgG3 | CPRCPAPELLGGPSVF | 6 | IgG2b | CHKCPAPNLEGGPSVF | 11 |
| IgG4 | AHHAQAPEFLGGPSVF | 7 | IgG3 | GSSCPAGNILGGPSVF | 9 |

The polypeptide preferably cleaves between the bold/underlined residues as shown for each of these sequences in Table 1. The cleavage site may alternatively be described as being between positions 249 and 250 of human IgG according to the Kabat numbering system (positions 236 and 237 according to EU numbering system).

The polypeptide preferably cleaves all human IgG subclasses, that is IgG1, IgG2, IgG3 and IgG4, and does not cleave other human immunoglobulin classes. The polypeptide may exhibit lower activity against human IgG2 as compared to the other human IgG subclasses. The polypeptide may exhibit greater activity against human IgG1 than human IgG2. The polypeptide may exhibit activity against non-human IgG molecules, including mouse IgG2a and IgG3. The polypeptide may exhibit activity against IgG from guinea pig or horse.

The polypeptide may efficiently cleave IgG to Fc and F(ab′)₂ fragments via a two-stage mechanism. In the first stage, one (first) heavy chain of IgG is cleaved to generate a single cleaved IgG (scIgG) molecule with a single non-covalently bound Fc chain. The scIgG molecule is effectively an intermediate product which retains the remaining (second) heavy chain of the original IgG molecule. The complex of a polypeptide of the invention and a single cleaved IgG molecule is also provided herein. In this context, the IgG cleaved by the polypeptide is preferably a recombinant, monoclonal antibody. Thus, a complex of a polypeptide of the invention and a single cleaved recombinant, monoclonal IgG molecule is provided. Exemplary antibodies that may be cleaved by the polypeptide of the invention are listed below in the “Methods of use” section. A complex of a polypeptide of the invention and a single cleaved version of any of these antibodies is provided.

In the second stage of the mechanism the remaining (second) heavy chain of the original IgG molecule is quickly cleaved by the polypeptide to release a F(ab′)₂ fragment and a homodimeric Fc fragment. These are the products generally observed under physiological conditions. The homodimeric Fc may dissociate into its component monomers. Under reducing conditions the F(ab′)2 fragment may dissociate to two Fab fragments.

Immunoglobulin protease activity may be assessed by any suitable method, for example by incubating a polypeptide with a sample containing IgG and determining the presence of IgG cleavage products. Assays for assessing immunoglobulin protease activity may also be used to quantify the efficacy of said activity, that is to assess the potency of a polypeptide. Efficacy may be assessed in the presence or absence of an inhibitor. However, efficacy herein will typically mean efficacy as assessed in the absence of such an inhibitor unless otherwise stated. Suitable assays to determine activity and/or quantify potency of said activity are well known in the art and any suitable assay may be used. Suitable assays include an ELISA-based assay. In such an assay, the wells of an assay plate will typically be coated with an antibody target, such as bovine serum albumin (BSA). Samples of the polypeptide to be tested are then added to the wells, followed by samples of target-specific antibody, that is antibody specific for BSA in this example. The polypeptide and antibody are allowed to interact under conditions suitable for IgG protease activity. After a suitable interval, the assay plate will be washed and a detector antibody which specifically binds to the target-specific antibody will be added under conditions suitable for binding to the target-specific antibody. The detector antibody will bind to any intact target-specific antibody that has bound to the target in each well. After washing, the amount of detector antibody present in a well will be proportional to the amount of target-specific antibody bound to that well. The detector antibody may be conjugated directly or indirectly to a label or another reporter system (such as an enzyme), such that the amount of detector antibody remaining in each well can be determined. The higher the potency of the tested polypeptide that was in a well, the less intact target-specific antibody will remain and thus there will be less detector antibody. In an embodiment, at least one well on a given assay plate will include mature IdeS and/or mature IdeZ and/or IdeSORK2.0 instead of a polypeptide to be tested, so that the potency of the tested polypeptides may be directly compared to the potency of mature IdeS and/or mature IdeZ and/or IdeSORK2.0.

Other assays may determine the potency of a tested polypeptide by directly visualizing and/or quantifying the fragments of IgG which result from cleavage of IgG by a tested polypeptide. Such an assay will typically incubate a sample of IgG with a test polypeptide (or mature IdeS and/or mature IdeZ and/or IdeSORK2.0 as a control) at differing concentrations in a titration series. The products which result from incubation at each concentration are then separated using gel electrophoresis, for example by SDS-PAGE. Whole IgG and the fragments which result from cleavage of IgG can then be identified by size and quantified by the intensity of staining with a suitable dye. The greater the quantity of cleavage fragments, the greater the potency of a tested polypeptide at a given concentration. This type of assay may also enable the identification of test polypeptides that are more effective at cleaving the first or the second heavy chain of an IgG molecule, as the quantities of the different fragments resulting from each cleavage event may also be determined. A polypeptide of the invention may be more effective at cleaving the first chain of an IgG molecule than the second chain.

A polypeptide of the invention is preferably at least as effective at cleaving human IgG1 as IdeSORK2.0 in an embodiment.

Suitable methods to assess immunoglobulin protease activity and efficacy are also described in the Examples.

### Structural Features of a Polypeptide Having Immunoglobulin Protease Activity

This section sets out the structural features of a polypeptide having immunoglobulin protease activity, which may apply in addition to the functional features outlined in the immediately preceding section. In particular, the structural features may apply in addition to the function of immunoglobulin protease activity.

The polypeptide is typically no longer than 600 amino acids in length. The polypeptide may comprise the 557 amino acids of SEQ ID NO: 1 and in addition may be engineered to include one or more additional amino acids upto a total length of 600 amino acids, wherein said additional amino acids are typically to assist with production, isolation or purification. The polypeptide may thus comprise, comprise, consist essentially, or consist of the sequence of SEQ ID NO: 1.

The polypeptide may have a maximum length of 560, 500, 400, or 350 amino acids.

The polypeptide preferably comprises, consists essentially of, or consists of any N terminal fragment of SEQ ID NO: 1, provided it has immunoglobulin protease activity. The polypeptide may comprise, consist essentially of, or consist of the first 150, 160, 170, 180, 190, 200, 210, 220, 230, 240, 250, 260, 270, 280, 290, 300, 301, 302, 303, 304, 305, 306, 307, 308, 309, 310, 311, 312, 313, 314, 315, 316, 317, 318 319, 320, 330, 340, 350, 360, 370, 380, 390, 400, 410, 420, 430, 440, 450, 460, 470, 480, 490 or 500 contiguous amino acids from the N terminus of SEQ ID NO: 1, provided that the polypeptide has immunoglobulin protease activity. The N-terminal fragment preferably comprises at least the first 250 contiguous amino acids from the N terminus of SEQ ID NO: 1, optionally up to the first 320 contiguous amino acids from the N terminus of SEQ ID NO: 1. An exemplary N terminal fragment of SEQ ID NO: 1 is provided as SEQ ID NO: 2. It comprises the first 312 contiguous amino acids from the N terminus of SEQ ID NO: 1.

Any N terminal fragment of SEQ ID NO: 1 may be engineered to include one or more additional amino acids, wherein said additional amino acids are typically to assist with production, isolation or purification. An engineered version of SEQ ID NO: 2 is provided as SEQ ID NO: 3. The immunoglobulin protease activity of the N terminal fragment is preferably at least 50% of the activity against human IgG of the polypeptide consisting of SEQ ID NO: 3, when measured in the same assay, and is more preferably comparable to or superior to the activity of the polypeptide consisting of SEQ ID NO: 3.

Alternatively, the polypeptide of the invention may comprise, consist essentially, or consist of a variant of the amino acid sequence of SEQ ID NO: 1, SEQ ID NO: 2, or any other N-terminal fragment of SEQ ID NO: 1 having immunoglobulin protease activity as defined above. The immunoglobulin protease activity of the variant polypeptide is preferably at least 50% of the activity against human IgG of the polypeptide consisting of SEQ ID NO: 3, when measured in the same assay, and is more preferably comparable to or superior to the activity of the polypeptide consisting of SEQ ID NO: 3.

Said variant may be at least 50% identical to the amino acid sequence of SEQ ID NO: 1, SEQ ID NO: 2, or said N-terminal fragment of SEQ ID NO:1. The variant sequence may be at least 60%, at least 70%, at least 75%, at least 80%, at least, 85%, at least 90%, at least 95%, at least 96%, at least 97%, at least 98% or at least 99% identical to the sequence of SEQ ID NO: 1, SEQ ID NO: 2, or said N-terminal fragment of SEQ ID NO:1. The identity level is preferably at least 85% or higher. Identity relative to the sequence of SEQ ID NO: 1, SEQ ID NO: 2, or said N-terminal fragment of SEQ ID NO:1 can be measured over a region of at least 150, 160, 170, 180, 190, 200, 210, 220, 230, 240, 250, 260, 270, 280, 290, 300, 301, 302, 303, 304, 305, 306, 307, 308, 309, 310, 311, 312, 313, 314, 315, 316, 317, 318 319, 320, 330, 340, 350, 360, 370, 380, 390, 400 or more contiguous amino acids of the sequence shown in SEQ ID NO: 1, SEQ ID NO: 2, or said N-terminal fragment of SEQ ID NO:1. More preferably, identity is measured over the full length of SEQ ID NO: 1, SEQ ID NO: 2, or said N-terminal fragment of SEQ ID NO:1. Identity to SEQ ID NO: 1, SEQ ID NO: 2, or said N-terminal fragment of SEQ ID NO: 1 may be measured over the length of the variant, provided the variant is of a length which is no more than 50 amino acids longer or shorter than the reference sequence, and is preferably of approximately (or exactly) the same length as the reference sequence. SEQ ID NO: 2 is the most preferred reference sequence. Identity of a variant is most preferably measured over the full length of SEQ ID NO: 2.

Amino acid identity may be calculated using any suitable algorithm. For example the PILEUP and BLAST algorithms can be used to calculate identity or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89:10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90:5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001. Alternatively, the UWGCG Package provides the BESTFIT program which can be used to calculate identity (for example used on its default settings) (Devereux et al (1984) Nucleic Acids Research 12, 387-395).

The sequence of a polypeptide of the invention may comprise a variant of the amino acid sequence of SEQ ID NO: 1, SEQ ID NO: 2, or any other N-terminal fragment of SEQ ID NO: 1 having immunoglobulin protease activity as defined above, in which modifications, such as amino acid additions, deletions or substitutions are made relative to the sequence of SEQ ID NO: 1, SEQ ID NO: 2, or said other N-terminal fragment of SEQ ID NO: 1.

Unless otherwise specified, the modifications are preferably conservative amino acid substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table A1 below. Where amino acids have similar polarity, this can be determined by reference to the hydropathy scale for amino acid side chains in Table A2. A sequence of a polypeptide of the invention may comprise a variant of the amino acid sequence of SEQ ID NO: 1 in which up to 10, 20, 30, 40, 50 or 60 conservative substitutions are made.

TABLE A1
Chemical properties of amino acids

| | | | |
|---|---|---|---|
| Ala (A) | aliphatic, hydrophobic, neutral | Met (M) | hydrophobic, neutral |
| Cys (C) | polar, hydrophobic, neutral | Asn (N) | polar, hydrophilic, neutral |
| Asp (D) | polar, hydrophilic, charged (−) | Pro (P) | hydrophobic, neutral |
| Glu (E) | polar, hydrophilic, charged (−) | Gln (Q) | polar, hydrophilic, neutral |
| Phe (F) | aromatic, hydrophobic, neutral | Arg (R) | polar, hydrophilic, charged (+) |
| Gly (G) | aliphatic, neutral | Ser (S) | polar, hydrophilic, neutral |
| His (H) | aromatic, polar, hydrophilic, charged (+) | Thr (T) | polar, hydrophilic, neutral |
| Ile (I) | aliphatic, hydrophobic, neutral | Val (V) | aliphatic, hydrophobic, neutral |
| Lys (K) | polar, hydrophilic, charged(+) | Trp (W) | aromatic, hydrophobic, neutral |
| Leu (L) | aliphatic, hydrophobic, neutral | Tyr (Y) | aromatic, polar, hydrophobic |

TABLE A2
Hydropathy scale

| | Hydropathy |
|---|---|
| Side Chain | scale |
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

A suitable modification may replace at least one amino acid in the starting sequence with an amino acid having comparable characteristics but which is not one of the 20 L-configuration amino acids appearing in natural eukaryotic proteins. For example, at least one L-configuration amino acid may be replaced with the directly corresponding non-L configuration amino acid, such as a D-configuration amino acid, or with a non-L configuration amino acid which is otherwise a conservative substitution as defined above. The amino acid sequence of a polypeptide of the invention may comprises a variant of the amino acid sequence as described above. However, certain residues are preferably retained within the said variant sequence. For example, numbering the positions starting from the N terminus of SEQ ID NO: 2, the Lys at position 66 is expected to be important for creating the oxyanion hole, the Cys as position 76 and the His at position 226 are predicted to constitute the active site, and the Asp amino acids at positions 248 and 250 are expected to be important for the proper orientation of the active site as well as creating an electrostatic milieu that promotes the hydrolysis. It is preferred that the amino acids in positions corresponding to these positions are not changed, particularly Cys-76 and His-226.

Any polypeptide of the invention which comprises SEQ ID NO:1, SEQ ID NO: 2, or any other N-terminal fragment of SEQ ID NO: 1 having immunoglobulin protease activity as defined above, or any variant of any one of these sequences, may optionally be engineered to include an additional methionine at the N terminus and/or protein purification or other tag at the C terminus. The tag is a sequence which is not naturally expressed in streptococcal bacteria as a contiguous domain of the IdeSORK protein of SEQ ID NO: 1. A preferred protein purification tag is a histidine tag. A histidine tag preferably consists of six histidine residues. The histidine tag may be linked to the C terminus by a linker, which is typically a short sequence of amino acids, such as 3-5 amino acids. The linker typically consists predominantly of glycine and serine residues, and may preferably include the sequence GSG. For example GSG and GSGLE are suitable linkers. SEQ ID NO: 3 is an example of an engineered sequence.

In summary therefore, provided herein is:

A polypeptide, optionally an engineered polypeptide, having immunoglobulin protease activity, wherein said polypeptide comprises, consists essentially of, or consists of:
(a) the amino acid sequence of SEQ ID NO: 1;
(b) the amino acid sequence of SEQ ID NO: 2;
(c) an amino acid sequence which is an N terminal fragment of the sequence of SEQ ID NO: 1;
(d) an amino acid sequence which is at least 50% identical to the amino acid sequence of any one of (a), (b) or (c);
optionally wherein said polypeptide is engineered to include an additional methionine at the N terminus and/or a protein purification or other tag at the C terminus, which tag may be joined to the C terminus by a linker.

SEQ ID NOs: 1, 2 and 3 are the sequences of exemplary polypeptides of the invention. The polypeptide may comprise, consist essentially, or consist of the amino acid sequence of any one of SEQ ID NOs: 1, 2 or 3. Exemplary polynucleotide sequences of the invention include SEQ ID NOs: 16 to 19.

### General Polypeptide Features

A “polypeptide” is used herein in its broadest sense to refer to a compound of two or more subunit amino acids, amino acid analogs, or other peptidomimetics. The term “polypeptide” thus includes short peptide sequences and also longer polypeptides and proteins. The terms “protein”, “peptide” and “polypeptide” may be used interchangeably. The term “amino acid” may refer to either natural and/or unnatural or synthetic amino acids, including both D or L optical isomers, and amino acid analogs and peptidomimetics.

A polypeptide may be produced by suitable method, including recombinant or synthetic methods. For example, the polypeptide may be synthesised directly using standard techniques known in the art, such as Fmoc solid phase chemistry, Boc solid phase chemistry or by solution phase peptide synthesis. Alternatively, a polypeptide may be produced by transforming a cell, typically a bacterial cell, with a nucleic acid molecule or vector which encodes said polypeptide. Production of polypeptides by expression in bacterial host cells is described below and is exemplified in the Examples. The invention provides nucleic acid molecules and vectors which encode a polypeptide of the invention. The invention also provides a host cell comprising such a nucleic acid or vector. Exemplary polynucleotide molecules encoding polypeptides disclosed herein are provided as SEQ ID NOs: 16 and 17. Each of these sequences includes at the 5′ end a codon for the N terminal methionine (ATG) and, prior to the stop codon (TAA) at the 3′ end, codons for a protein purification tag, in this case 6×His tag, which may optionally be excluded. The optional inclusion of an additional methionine and a tag are discussed in more detail elsewhere in this document. SEQ ID NOs: 18 and 19 are expression vector sequences comprising SEQ ID NOs: 16 and 17, respectively.

The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention encodes a polypeptide of the invention and may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated. A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences, for example in an expression vector. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Polynucleotides can be synthesised according to methods well known in the art, as described by way of example in Sambrook et al (1989, Molecular Cloning—a laboratory manual; Cold Spring Harbor Press). The nucleic acid molecules of the present invention may be provided in the form of an expression cassette which includes control sequences operably linked to the inserted sequence, thus allowing for expression of the polypeptide of the invention in vivo (e.g. in prokaryotic or eukaryotic expression systems). These expression cassettes, in turn, are typically provided within vectors (e.g., plasmids or recombinant viral vectors). Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art. By way of further example in this regard we refer to Sambrook et al.

The invention also includes cells that have been modified to express a polypeptide of the invention. Such cells typically include prokaryotic cells such as bacterial cells, for example E. coli. Such cells may be cultured using routine methods to produce a polypeptide of the invention.

A polypeptide may be engineered or modified to assist with production, isolation or purification. For example, where a polypeptide of the invention is produced by recombinant expression in a bacterial host cell, the sequence of the polypeptide may include an additional methionine (M) residue at the N terminus to improve expression. As another example, the polypeptide of the invention may be engineered or modified by addition of protein purification tag and the N or C terminus, preferably at the C terminus. The protein purification tag is preferably a moiety which is not naturally expressed in streptococcus bacteria. The protein purification tag is preferably a moiety which is not present in a wildtype polypeptide chain as expressed in streptococcus bacteria. A protein purification tag may be a ligand which is capable of binding directly and specifically to a separation means. Alternatively, the protein purification tag may be one member of a binding pair and the separation means comprises a reagent that includes the other member of the binding pair. Any suitable binding pair can be used.

Where the polypeptide is engineered or modified by addition of one member of a binding pair, the polypeptide is preferably histidine-tagged or biotin-tagged. Typically the amino acid coding sequence of the histidine or biotin tag is included at the gene level and the polypeptide is expressed recombinantly in E. coli. The histidine or biotin tag is typically present at either end of the polypeptide, preferably at the C-terminus. It may be joined directly to the polypeptide or joined indirectly by any suitable linker sequence, such as 3, 4 or 5 glycine residues, or a mixture of glycine and serine residues. The histidine tag typically consists of six histidine residues, although it can be longer than this, typically up to 7, 8, 9, 10 or 20 amino acids or shorter, for example 5, 4, 3, 2 or 1 amino acids.

Alternative tags useful for protein purification include Maltose-binding protein (MBP), glutathione S-transferase (GST), thioredoxin (TRX), ubiquitin (Ub), small ubiquitin related modifier (SUMO), solubility-enhancer peptide sequences (SET), and N-utilization substance (NusA). Suitable tags are also discussed in Costa et al; Front Microbiol. 2014; 5:63, which is incorporated by reference, including in particular each of the tags that are disclosed in Table 1 of Costa et al.

The amino acid sequence of a polypeptide may be modified or engineered to include at least one non-naturally occurring amino acid, for example to increase stability. When the polypeptides are produced by synthetic means, such amino acids may be introduced during production. The polypeptides may also be modified following either synthetic or recombinant production. Polypeptides may also be produced using D-amino acids. In such cases the amino acids will be linked in reverse sequence in the C to N orientation. This is conventional in the art for producing such polypeptides. Preferred polypeptides of the invention may be engineered to include at least one such unnatural or synthetic amino acid, or at least one non-L configuration amino acid.

A number of side chain modifications are known in the art and may be made to the side chains of the polypeptides, subject to the polypeptides retaining any further required activity or characteristic as may be specified herein. It will also be understood that polypeptides may be chemically modified, e.g. post-translationally modified. For example, they may be glycosylated, phosphorylated or comprise modified amino acid residues. The polypeptide may be PEGylated.

A polypeptide may be provided in a substantially isolated or purified form. That is, isolated from the majority of the other components present in a cellular extract from a cell in which the polypeptide was expressed. The polypeptide may be mixed with carriers or diluents (as discussed below) which will not interfere with the intended use and still be regarded as substantially isolated. It may also be in a substantially purified form, in which case it will generally comprise at least 90%, e.g. at least 95%, 98% or 99%, of the protein in the preparation. Where a polypeptide is provided in a composition with an additional active component, such as another polypeptide, each said polypeptide will individually be purified to a high level of homogeneity prior to mixing in an appropriate ratio for the intended purpose of each. For example, two polypeptides may be each be purified to at least 90% homogeneity prior to combining in a 1:1 ratio. Exemplary polypeptides that may be included in a mixture with a polypeptide of the invention may include any one or more of: an exoglycosidase; an endoglycosidase (e.g. EndoS, EndoS2, EndoE, including any polypeptide with endoglycosidase activity as disclosed in WO2013/037824); a protease (preferably an immunoglobulin protease with different specificity to the polypeptide of the invention, e.g. a protease as disclosed in WO2017/134274-such as a polypeptide comprising SEQ ID NO: 3 of that document or a variant thereof as disclosed in that document); or an amidase (e.g. PngaseF).

A polypeptide may be provided in lyophilised form, suitable for reconstitution in aqueous solution prior to use. The lyophilised composition has improved stability enabling longer storage of the polypeptide. A polypeptide is typically substantially purified prior to freeze-drying. A method of preparing a polypeptide in lyophilised form, comprising freeze-drying said polypeptide in a suitable buffer, such as Phosphate-buffered saline (PBS), Tris-buffered saline (TBS), or another Tris-buffer is provided herein. The resulting polypeptide in lyophilised form is also provided. A method of preparing a solution of a polypeptide, comprising providing the polypeptide in lyophilised form and reconstituting with a suitable carrier or diluent, such as water, is also provided.

A polypeptide may be immobilised using methods known in the art, for example as described in Datta S et al., Enzyme immobilization: an overview on techniques and support materials, 3 Biotech, 3 (1): 1-9 (2013). For example, the polypeptide may be immobilised by adsorption, covalent binding, affinity immobilization or entrapment. Materials that can be used as supports include but are not limited to for example, natural supports such as agarose, sepharose, collagen, gelatin, cellulose, pectin, sepharose, inorganic materials such as ceramics, silica, glass, activated carbon or charcoal, or synthetic polymers, such as Poly(styrene-divinylbenzene), or latex. Any of these may be provided as a resin or in any other suitable format. The polypeptide may be immobilised on magnetic beads.

### Compositions and Formulations Comprising Polypeptides

In another aspect, the present invention provides compositions comprising a polypeptide of the invention. For example, the invention provides a composition comprising one or more polypeptides of the invention and at least one excipient, preferably a preservative or stabiliser, and optionally also one or more additional carriers, diluents or vehicles. Exemplary classes of excipient and individual examples are included in the following table.

| Excipient | Examples |
|---|---|
| Salts | Ammonium sulfate, calcium chloride, magnesium sulfate, magnesium chloride, |
| | potassium chloride, sodium chloride, sodium gluconate, sodium sulfate, zinc |
| | chloride |
| Buffers | Acetate, carbonate, citrate, citrate-phosphate, HEPES, histidine, maleate, |
| | phosphate, succinate, tartrate, triethanolamine (Tris) |
| Sugars and polyols | Cyclodextrins, fructose, glucose, glycerol, inositol, lactose, maltose, mannitol, |
| | sorbitol, sucrose, trehalose |
| Amino acids | Alanine, arginine, aspartic acid, glycine, lysine, proline |
| Surfactants | Poloxamer 188/407, polysorbate 20/40/80, sodium lauryl sulfate |
| Antioxidants and | Ascorbic acid, benzyl alocohol, benzoic acid, citric acid, chlorobutanol, m-cresol, |
| preservatives | glutathione, methionine, methylparaben, phenol, propylparaben, sodium sulphite |
| Polymers | Dextran, polyethylene glycol |
| Other | Albumin, dimethyl sulfoxide, EDTA, ehanol, thioglycolic acid |

Typically, the final composition is sterile and pyrogen free.

In a particular embodiment, all of the present excipients are preferably pharmaceutically acceptable, in the sense of being compatible with the other ingredients of the composition and not deleterious to a subject to which the composition is administered. In this case, the composition may be referred to as a pharmaceutical composition.

Formulation of a suitable composition can be carried out using standard pharmaceutical formulation chemistries and methodologies all of which are readily available to the reasonably skilled artisan. For example, the agent can be combined with a preservative, and one or more carriers, excipients or vehicles. Auxiliary substances, such as wetting or emulsifying agents, pH buffering substances, reducing agents and the like, may be present in the excipient or vehicle. Suitable reducing agents include cysteine, thioglycerol, thioreducin, glutathione and the like. Excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as polyethyleneglycol, hyaluronic acid, glycerol, thioglycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

Such compositions may be prepared, packaged, or sold in a form suitable for bolus administration or for continuous administration. Injectable compositions may be prepared, packaged, or sold in unit dosage form, such as in ampoules or in multi-dose containers containing a preservative. Compositions include, but are not limited to, suspensions, solutions, emulsions in oily or aqueous vehicles, pastes, and implantable sustained-release or biodegradable formulations. Such compositions may further comprise one or more additional ingredients including, but not limited to, suspending, stabilizing, or dispersing agents. In one embodiment of a composition for parenteral administration, the active ingredient is provided in dry (for e.g., a powder or granules) form for reconstitution with a suitable vehicle (e. g., sterile pyrogen-free water) prior to parenteral administration of the reconstituted composition. The compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Other parentally-administrable compositions which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt. The compositions may be suitable for administration by any suitable route including, for example, intradermal, subcutaneous, percutaneous, intramuscular, intra-arterial, intraperitoneal, intraarticular, intraosseous or other appropriate administration routes. Preferred compositions are suitable for administration by intravenous infusion.

### Methods of Use of Polypeptides

The polypeptides, polynucleotides, expression vectors and/or compositions of the invention are useful in various methods. Thus, provided herein are methods comprising administering a polypeptide, polynucleotide, expression vector and/or composition of the invention, for example to a sample or a subject. Also provided are methods comprising contacting IgG with a polypeptide, polynucleotide, expression vector or composition of the invention. The method may be a method of cleaving an immunoglobulin, and may optionally further comprises the detection or analysis of the cleavage products. The method may be a method treating a disease, the method comprising administering a polypeptide of the invention to a subject.

For example, the present polypeptides may provide useful tools for biotechnology. The polypeptides may be used in a method for the ex vivo cleavage of IgG. The IgG may be human, mouse, horse or guinea pig IgG. The IgG may be a recombinant, monoclonal antibody derived from any of these species. The polypeptide may be administered to a sample containing IgG and incubated under conditions which permit immunoglobulin protease activity to occur.

Suitable conditions include incubation for at least 20 minutes, 30 minutes, 40 minutes, 50 minutes, 60 minutes, 70 minutes, 80 minutes, 90 minutes or 120 minutes, 3 hours, 5 hours, 10 hours, or overnight, typically with mixing e.g. end-over-end mixing. A preferred incubation time is around 30 minutes or around 1 hour.

Incubation preferably takes place at room temperature, more preferably at approximately 20° C., 25° C., 30° C., 35° C., 40° C. or 45° C., and most preferably at approximately 37° C.

The methods described above may be carried out under any suitable pH. Suitable pH values include, for example, around pH 6.5 to around pH 8.5, preferably around pH 7.0 to around pH 8.5, most preferably at around pH 7.5.

The method may be conducted in any suitable buffer, such as tris buffered saline (TBS) or phosphate buffered saline (PBS). There is preferably no requirement for salts, co-factors or reducing agents, although the presence of salts (e.g. NaCl) at up to 200 mM is preferably tolerated by the polypeptide of the invention.

The approximate ratio of the polypeptide of the invention to the protein content of the sample may be 1:1, 2:1, 4:1, 6:1, 10:1, 15:1, 20:1, 1:2, 1:4, or 1:6, 1:10, 1:15, 1:20, 1:40, 1:100, 1:200, 1:400, 1:500, 1:600, 1:700, 1:800, 1:900, 1:1000, 1:1500, 1:2000, or up to 1:2500 (wt:wt). A preferred ratio is between 1:20 and 1:40 (wt:wt).

Specific cleavage can be verified, and the cleavage products isolated using any suitable method, such as those described elsewhere in this document, including in the Examples, or in WO2003051914 and WO2009033670. The method can be used in particular to generate Fc and F(ab′)₂ fragments. Fab fragments may then be produced by carrying out a reduction step (for example in 2-mercaptoethanolamine or Cysteamine) on the F(ab′)₂ fragments that result from cleavage of IgG with a polypeptide of the invention.

The method may also be used to detect or analyse IgG in a sample, or to remove IgG from a sample. A method for the detection of IgG in a sample typically involves incubating the polypeptide with the sample under conditions which permit binding and cleavage. The presence of IgG can be verified by detection of the specific IgG cleavage products, which may subsequently be analysed.

The polypeptides, polynucleotides, expression vectors and/or compositions may also be used in therapy or prophylaxis. In therapeutic applications, polypeptides or compositions are administered to a subject already suffering from a disorder or condition, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as “therapeutically effective amount”. In prophylactic applications, polypeptides or compositions are administered to a subject not yet exhibiting symptoms of a disorder or condition, in an amount sufficient to prevent or delay the development of symptoms. Such an amount is defined as a “prophylactically effective amount”. The subject may have been identified as being at risk of developing the disease or condition by any suitable means. Thus the invention also provides a polypeptide of the invention for use in the treatment of the human or animal body. Also provided herein is a method of prevention or treatment of disease or condition in a subject, which method comprises administering a polypeptide of the invention to the subject in a prophylactically or therapeutically effective amount. The polypeptide is preferably administered by intravenous infusion, but may be administered by any suitable route including, for example, intradermal, subcutaneous, percutaneous, intramuscular, intra-arterial, intraperitoneal, intraarticular, intraosseous or other appropriate administration routes. The amount of said polypeptide that is administered may be at least about 0.01 mg/kg BW, at least about 0.05, 0.1, 0.15, 0.2, 0.2 or 0.25 mg/kg BW, up to at most about 0.5 mg/kg BW, at most about 1.0, 1.5, 2.0, or 2.5 mg/kg BW. Any of the above lower limits may be combined with any of the upper limits to provide a dose range. The amount is preferably between 0.2 and 1.2 mg/kg BW.

Polypeptides of the invention may be particularly useful in a method for the treatment or prevention of a disease or condition mediated by IgG antibodies, which in this context may be described as pathogenic IgG antibodies. Accordingly, the invention provides a polypeptide of the invention for use in the treatment or prevention of a disease or condition, particularly a disease or condition mediated by pathogenic IgG antibodies. The invention also provides a method of treating or preventing a disease or condition, particularly a disease or condition mediated by pathogenic IgG antibodies, comprising administering to an individual a polypeptide of the invention. The method may comprise repeat administration of the said polypeptide. The invention also provides a polypeptide of the invention for use in the manufacture of a medicament for the treatment or prevention of a disease or condition mediated by pathogenic IgG antibodies, particularly a disease or condition mediated by pathogenic IgG antibodies.

Numerous autoimmune diseases are mediated in whole or in part by pathogenic IgG antibodies. Thus, pathogenic antibodies may typically be specific for an antigen which is targeted in an autoimmune disease or other condition mediated wholly or in part by IgG antibodies. Exemplary diseases and conditions mediated by antibodies and the associated antigens are listed in detail in Table D of WO 2016/128559. A polypeptide of the invention may be used in a method to treat any of these diseases or conditions.

Pathogenic antibodies may recognise a tissue or organ transplant received by the subject. Thus the disease treated or prevented by the polypeptide may be antibody mediated transplant rejection. The pathogenic antibodies may recognise another therapeutic agent administered to the subject, such as an antibody, a gene therapy (e.g. a viral or other vector), a replacement for a defective endogenous factor such as an enzyme, a growth or a clotting factor, or a cell therapy. Thus, the disease or condition treated or prevented by the polypeptide may be any antibody response of the subject which reduces the efficacy or benefit to the subject of said therapeutic agent.

Polypeptides of the invention may be particularly useful for the treatment or prevention of a disease or condition in a subject, wherein the subject comprises pre-existing immunity to other IgG proteases. For example, the subject may comprise antibodies against IdeS or IdeZ, due to a previous S. pyogenes infection or prior treatment with IgG protease.

Methods of the invention may involve cleavage of a sample containing a known antibody. The antibody is preferably a recombinant monoclonal antibody. The antibody may be Abagovomab, Abciximab, Actoxumab, Adalimumab, Adecatumumab, Afelimomab, Afutuzumab, Alacizumab pegol, ALD518, Alemtuzumab, Alirocumab, Altumomab pentetate, Amatuximab, Anatumomab mafenatox, Anrukinzumab, Apolizumab, Arcitumomab, Aselizumab, Atinumab, Atlizumab (=tocilizumab), Atorolimumab, Bapineuzumab, Basiliximab, Bavituximab, Bectumomab, Belimumab, Benralizumab, Bertilimumab, Besilesomab, Bevacizumab, Bezlotoxumab, Biciromab, Bimagrumab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Capromab pendetide, Carlumab, Catumaxomab, CC49, Cedelizumab, Certolizumab pegol, Cetuximab, Ch.14.18, Citatuzumab bogatox, Cixutumumab, Clazakizumab, Clenoliximab, Clivatuzumab tetraxetan, Conatumumab, Concizumab, Crenezumab, CR6261, Dacetuzumab, Daclizumab, Dalotuzumab, Daratumumab, Demcizumab, Denosumab, Detumomab, Dorlimomab aritox, Drozitumab, Duligotumab, Dupilumab, Dusigitumab, Ecromeximab, Eculizumab, Edobacomab, Edrecolomab, Efalizumab, Efungumab, Elotuzumab Elsilimomab, Enavatuzumab, Enlimomab pegol, Enokizumab, Enoticumab, Ensituximab, Epitumomab cituxetan, Epratuzumab, Erlizumab, Ertumaxomab, Etaracizumab, Etrolizumab, Evolocumab, Exbivirumab, Fanolesomab, Faralimomab Farletuzumab, Fasinumab, FBTA05, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, GS6624, Ibalizumab, Ibritumomab tiuxetan, Icrucumab, Igovomab, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lampalizumab, Lebrikizumab, Lemalesomab, Lerdelimumab, Lexatumumab, Libivirumab, Ligelizumab, Lintuzumab, Lirilumab, Lodelcizumab, Lorvotuzumab mertansine, Lucatumumab, Lumiliximab, Mapatumumab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Obinutuzumab, Ocaratuzumab, Ocrelizumab, Odulimomab, Ofatumumab, Olaratumab, Olokizumab, Omalizumab, Onartuzumab, Oportuzumab monatox, Oregovomab, Orticumab, Otelixizumab, Oxelumab, Ozanezumab, Ozoralizumab, Pagibaximab, Palivizumab, Panitumumab, Panobacumab, Parsatuzumab, Pascolizumab, Pateclizumab, Patritumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Teneliximab, Teplizumab, Teprotumumab, TGN1412, Ticilimumab (=tremelimumab), Tildrakizumab, Tigatuzumab, TNX-650, Tocilizumab (=atlizumab), Toralizumab, Tositumomab, Tralokinumab, Trastuzumab, TRBS07, Tregalizumab, Tremelimumab Tucotuzumab celmoleukin, Tuvirumab, Ublituximab, Urelumab, Urtoxazumab, Ustekinumab, Vapaliximab, Vatelizumab, Vedolizumab, Veltuzumab, Vepalimomab Vesencumab, Visilizumab, Volociximab, Vorsetuzumab mafodotin, Votumumab, Zalutumumab, Zanolimumab, Zatuximab, Ziralimumab or Zolimomab aritox.

Polypeptides of the invention may be useful in a method for improving the benefit to a subject of a therapeutic antibody or therapeutic agent, such as whose effect is mediated through Fc receptor binding. For example, the polypeptides of the invention may rapidly, temporarily and safely eliminate Fc receptor binding by all or substantially all endogenous IgG in a subject. Thus, an administered, such as a subsequently administered, therapeutic antibody (or therapeutic agent, such as that binds Fc receptor) would have enhanced efficacy because it does not need to compete with endogenous IgG for binding to Fc receptor.

The following Examples illustrate the invention.

### Example 1

Unless indicated otherwise, the methods used are standard biochemistry and molecular biology techniques. Examples of suitable methodology textbooks include Sambrook et al., Molecular Cloning, A Laboratory Manual (1989) and Ausubel et al., Current Protocols in Molecular Biology (1995), John Wiley and Sons, Inc.

### Analysis of New Protease

A study was performed upon which many different rodents (moles and voles) were caught, and streptococcal species thereof were isolated and partly characterized. One of these isolates, tentatively called Streptococcus krösus, was sequenced and was found to have several glycosidases and proteases. One gene in particular had a low degree of homology to IdeS. This gene, referred to herein as IdeSORK, is characterised below.

Initial Identification of Bacteria from Wild Voles

As a part of a larger ecological project, wild voles (Microtus arvalis) in the south of Sweden were throat swabbed and released again. The swabs were cultured on 4% horse blood agar plates and were serially re-streaked to obtain pure isolates. Colony color and morphology and hemolytic (a, B or y) pattern was recorded. A total of 57 different isolates were analyzed of which the 8 clearly β hemolytic ones where chosen for further analysis.

### Phylogenetic Analysis of Isolates

### 16S DNA Sequencing

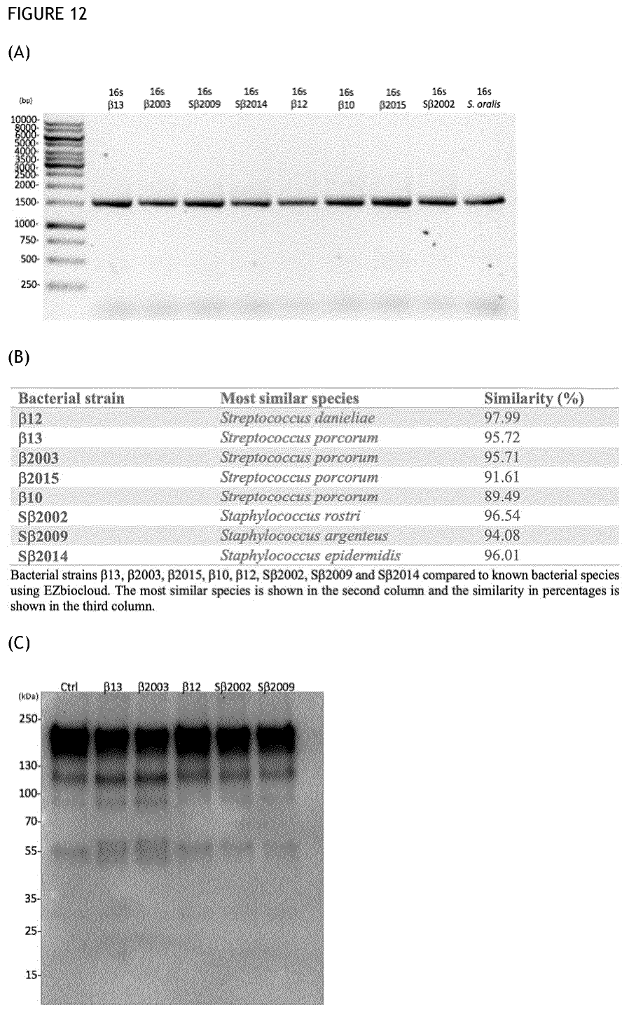

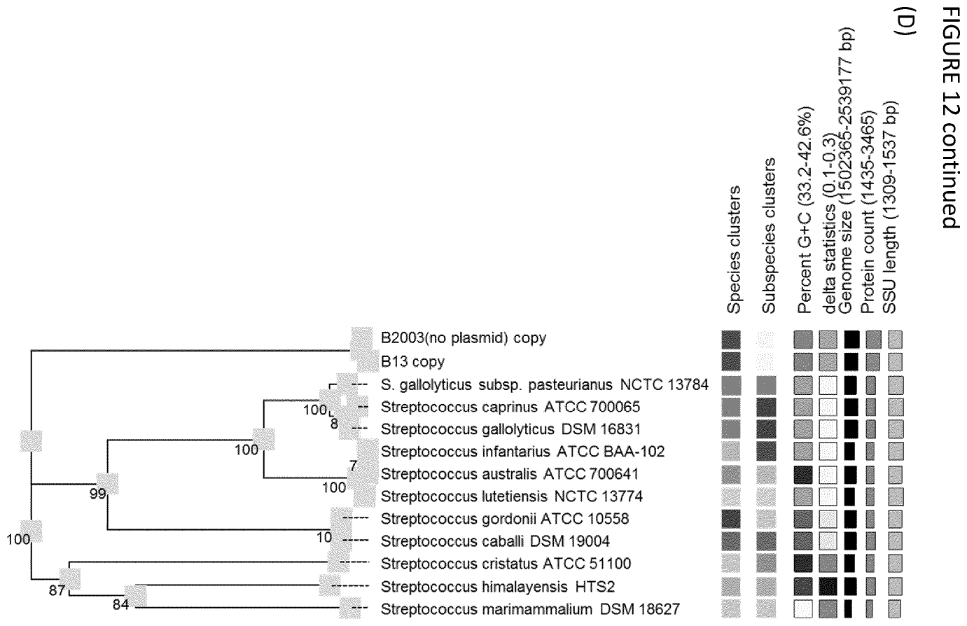

1541 base pair PCR products for the 16S gene were generated from all 8 strains (and a known control strain of Streptococcus oralis) (FIG. 12A). Products were sequenced in full using Sanger sequencing and were queried against databases with known 16S sequences (FIG. 12B).

### Metabolic Characterization

All isolates were subjected to metabolic testing using Streptococcal and Staphylococcal tests. Results were inconclusive, indicating that all strains are metabolically distinct from known species (positive controls for Streptococcus pyogenes, Streptococcus oralis and Staphylococcus aureus were correctly identified).

### Activity Testing on Mouse IgG

All isolates were screened for protelytic activity on mouse IgG. β13 and β2003 showed weak activity on IgG and were therefore chosen for further analysis (FIG. 12C).

### Whole Genome Sequencing of β13 and β2003

Genomic DNA from β13 and β2003 where sequenced using a combination of Oxford Nanopore (ONT) and Illumina sequencing. De novo assembly of ONT long reads was done using the assembler Canu. This resulted in that both genomes could be assembled into one draft genome contig. β2003 also contained two extrachromosomal plasmids. Correction was done using Nanoplish and the Illumina reads were mapped onto the draft contigs. Gene annotation were made using RAST. Whole genome of phylogeny the two approximately 2.46 Mb genomes confirmed that both strains are most likely distinct isolates of the same new species (FIG. 12D). When analyzing the annotated genomes in detail the gene encoding the putative IgG endopeptidase could be found in β13 but not in β2003.

### CONCLUSIONS

Several new streptococcal and staphylococcal species could be identified in the throats of wild voles. The β hemolytical ones β13 and β2003 where quite surprisingly closely related to oral streptococci that normally are a hemolytic. No IgG protease have to date been found in any of the oral streptococci or close relatives, so the finding of the IgG protease Xork in these strains was quite unexpected.

### Production and Purification

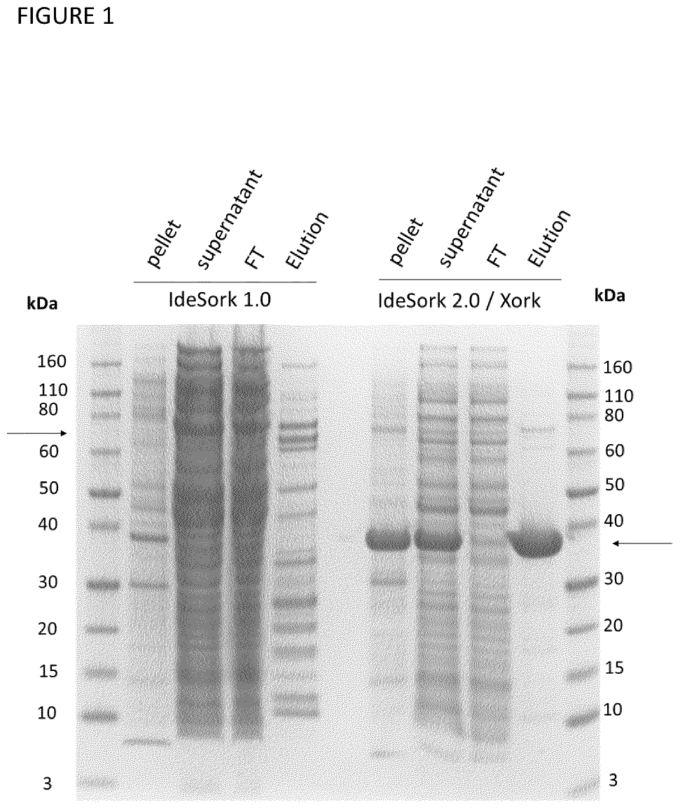

The full length protein of SEQ ID NO: 1 (IdeSORK) consistently expressed poorly and to a low purity (FIG. 1). However, a truncated version lacking a ca 20 kDa long C-terminal Domain of SEQ ID NO: 1, engineered with N terminal M and C terminal His tag (IdeSORK2.0; SEQ ID NO: 3) behaves well, and can be expressed in E. coli BL21 (DE3) STAR cells as a soluble protein with high purity and rather high expression. The expression and purification follows standard protocols, culturing cells at 37° C., inducing using 1 mM IPTG, and harvesting cells after 5 hours of induction. When purifying (GraviTRAP) material after sonication, we often get a yield around 5 mg/g. Modifying the temperature to 30° C. during induction can significantly improve the production, reaching ca 9 mg/g (data not shown).

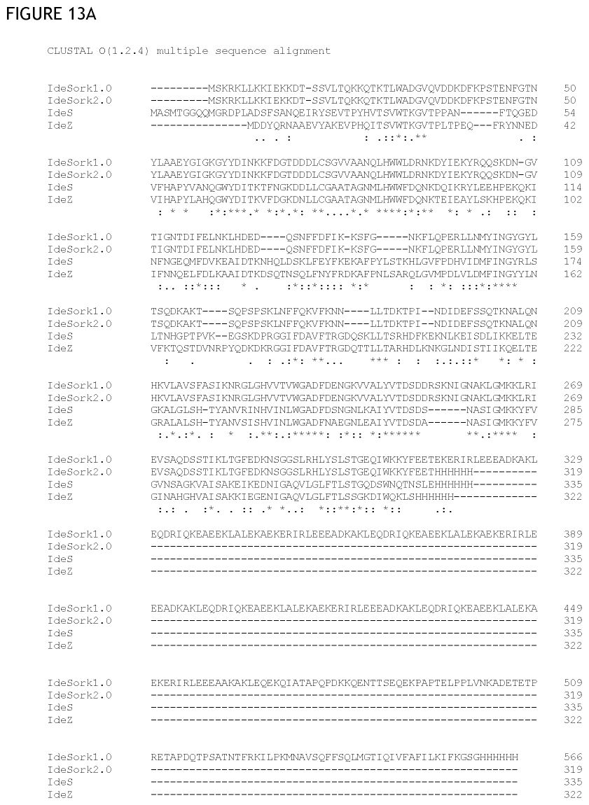

Further truncation of the construct results in very low yields and low activity, while additions to the C-terminus (relative to IdeSORK 2.0) is more accepted, but partly affects yield and activity (FIG. 13B).

### Activity

### Specificity

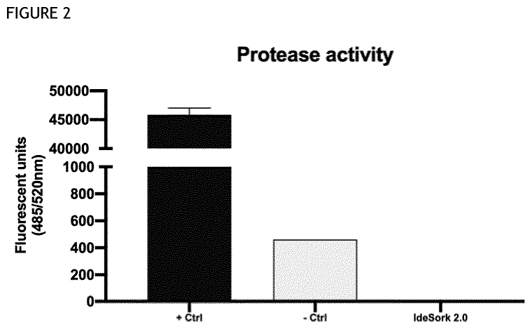

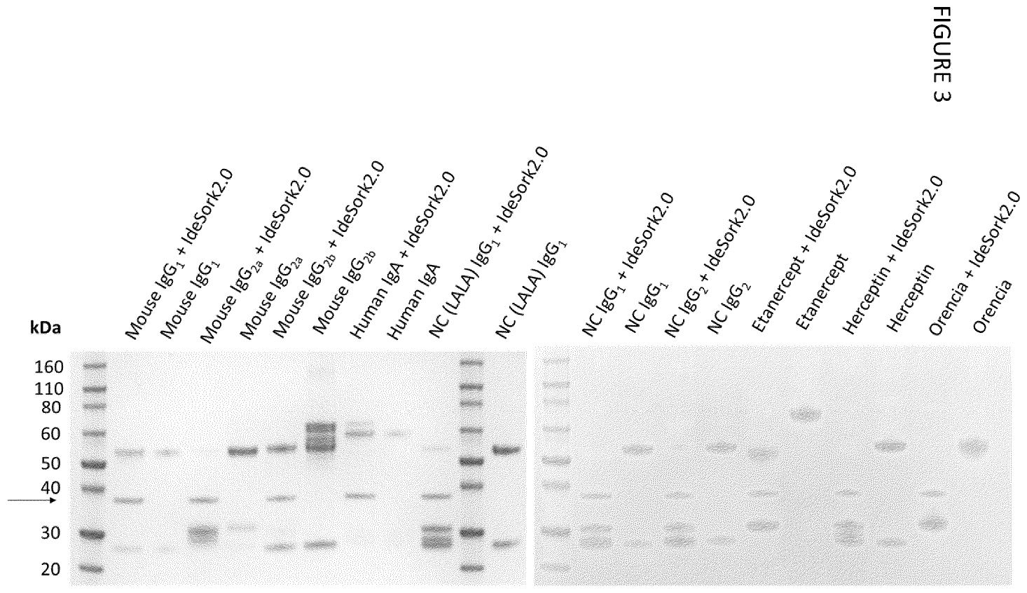

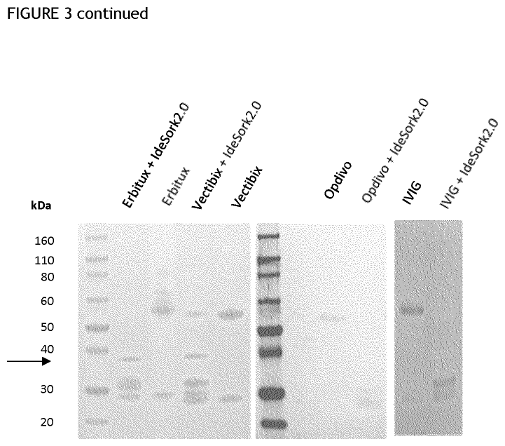

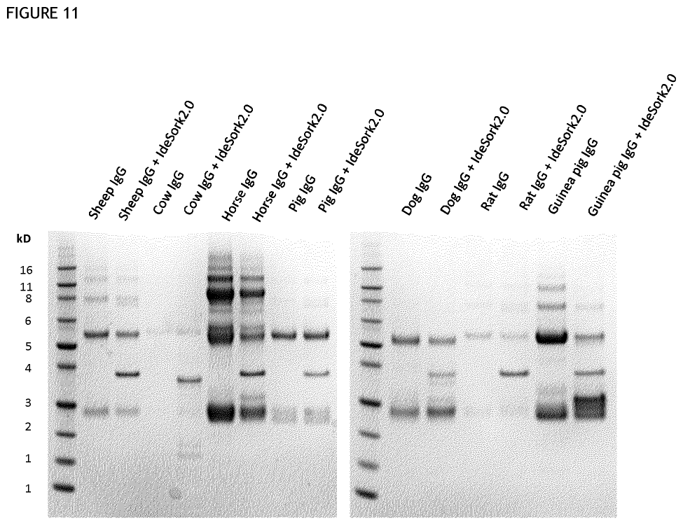

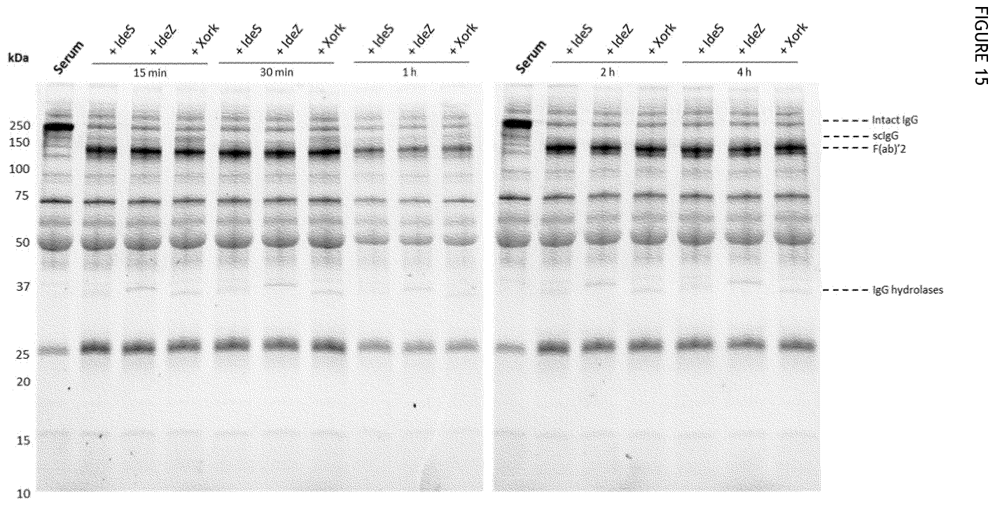

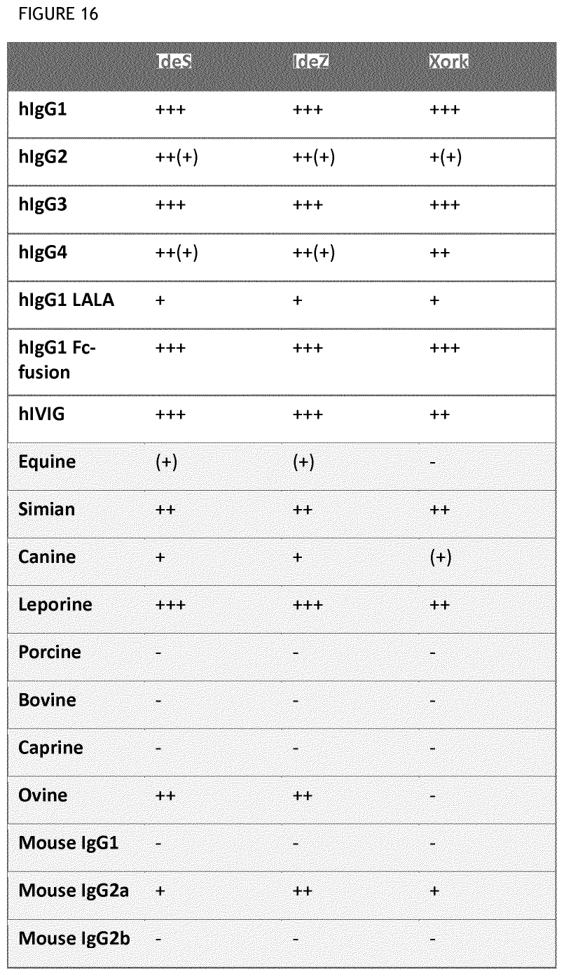

Unlike IdeS (−Ctrl), IdeSORK2.0 shows no signs of general protease activity towards casein/gelatin (FIG. 2), while hydrolyzing all tested human IgGs and IgG Fc fusion proteins (FIG. 3 and FIG. 16). IdeSORK2.0 is not active towards IgA, indicating that it is not the general immunoglobulin structure that dictates activity, but rather an even higher specificity (FIG. 3). The enzyme has a slight preference for IgG1 over IgG2, similar to IdeS, and can also hydrolyze monoclonal IgG4. For mouse IgG, the protease is active against mIgG2a, but not mIgG1 or mIgG2b. Alterations of the hinge region of human antibodies (e.g. LALA-sequence) did not affect the activity of IdeSORK2.0. Incubation with polyclonal IgG (e.g. IVIG) resulted in near complete hydrolysis indicating that the enzyme is not limited to specific monoclonal antibodies but can hydrolyze all human IgG. A further examination towards more animal IgG (porcine, horse, dog, rat, guinea pig, sheep, cow) only showed activity against horse and guinea pig IgG (FIG. 11).

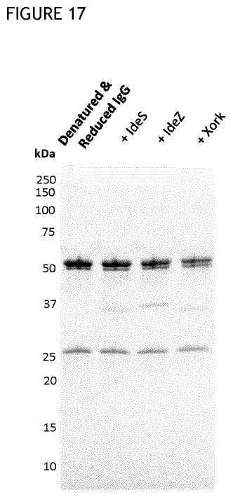

IdeSORK2.0 is not active against denatured and reduced IgG (FIG. 17).

### Protease Inhibitor Sensitivity

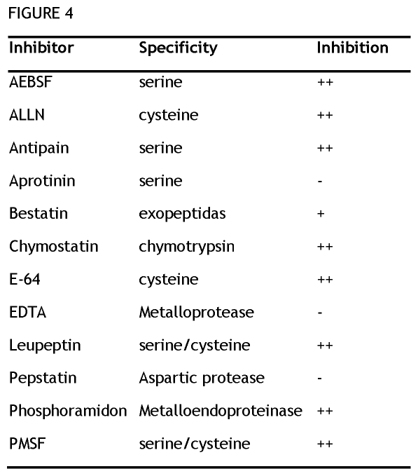

IdeS is characterized as a cysteine protease, and is as such not affected by most protease inhibitors other than E64. The enzymatic mechanism of the new protease identified here has not been fully characterized, but IdeSORK2.0 is highly sensitive for many protease inhibitors, including known serine and cysteine protease inhibitors (FIG. 4). The enzyme does however not depend on cations, and is unaffected by presence of EDTA (FIG. 4).

### Site of Hydrolysis

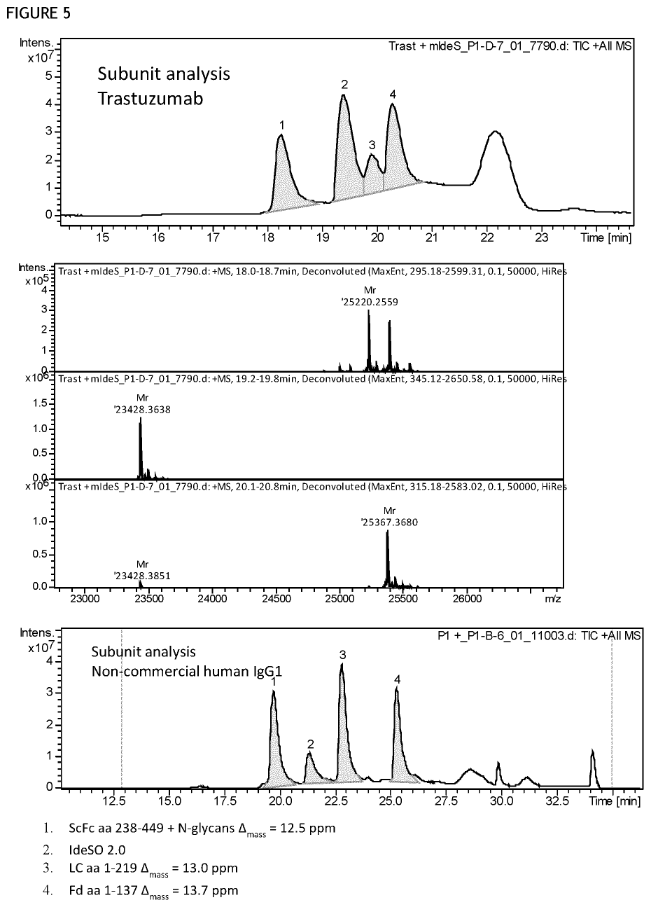

Several substrates (IgG1, IgG2, fusion proteins, LALA-mutated IgG) were subjected to hydrolysis by IdeSORK2.0 and the site of hydrolysis identified through LC-MS. As shown in FIG. 5, only a single hydrolytic site was detected, being identical in all sequences, and corresponding to the human IgG1 hinge/CH2 region (see Table 1 above and also the table below). This is the same site that is recognized by IdeS. The site of hydrolysis has been confirmed for etanercept, trastuzumab, cetuximab, as well as two non-commercial monoclonal antibodies.

| Name | Description | Digestion site |
|---|---|---|
| Non-commercial mAb | Human IgG1 | CPPCPAPELLG / |
| | | GPSVFLFPPKP |
| Trastuzumab | Human IgG1 | CPPCPAPELLG / |
| | | GPSVFLFPPKP |
| Non-commercial mAb | Human IgG1 LALA modified | CPPCPAPEAAG / |
| | | GPSVFLFPPKP |
| Cetuximab | Chimeric human /mouse IgG1 | CPPCPAPELLG / |
| | | GPSVFLFPPKP |
| Etanercept | Dimeric human IgG1 -TNFR fusion protein | CPPCPAPELLG / |
| | | GPSVFLFPPKP |

### Mechanism of Action

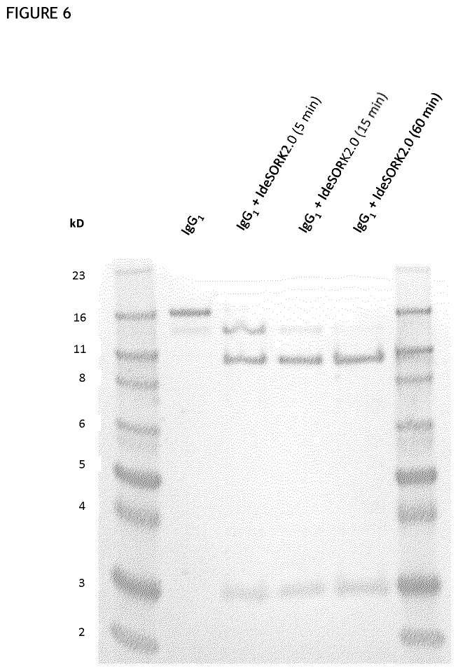

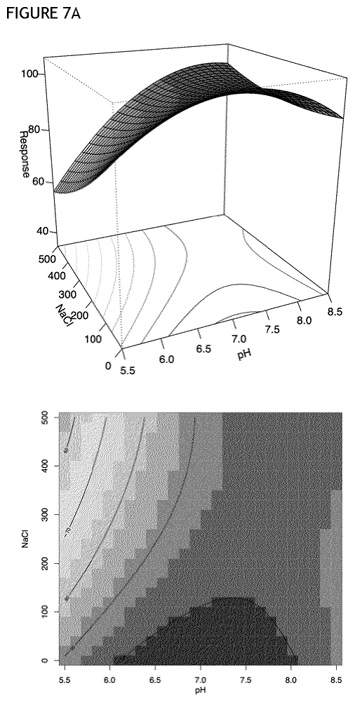

IdeS is known to first hydrolyze one chain of the antibody, and upon hydrolysis thereof, have lower affinity to the second chain. As such many antibodies may be only partly hydrolyzed at the start of the reaction. IdeSORK2.0 also generates fragments with single chain cleavage, however a large proportion of these are quickly processed to full cleavage (FIG. 6). The affinity for the second chain has not been determined, but it is clear that activity is mediated one chain at a time.

### Optimal Conditions

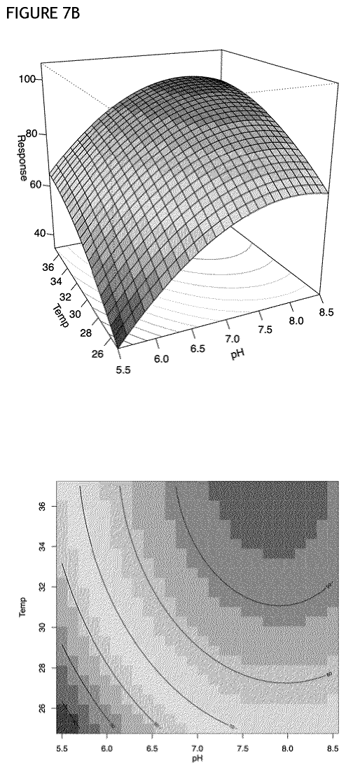

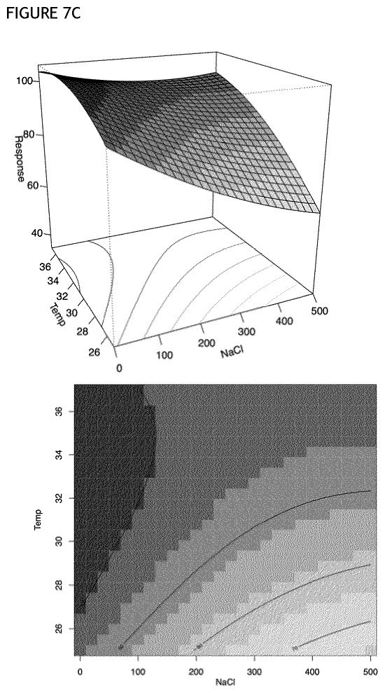

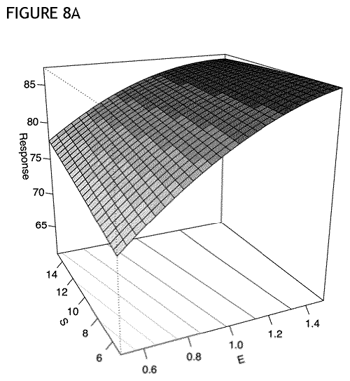

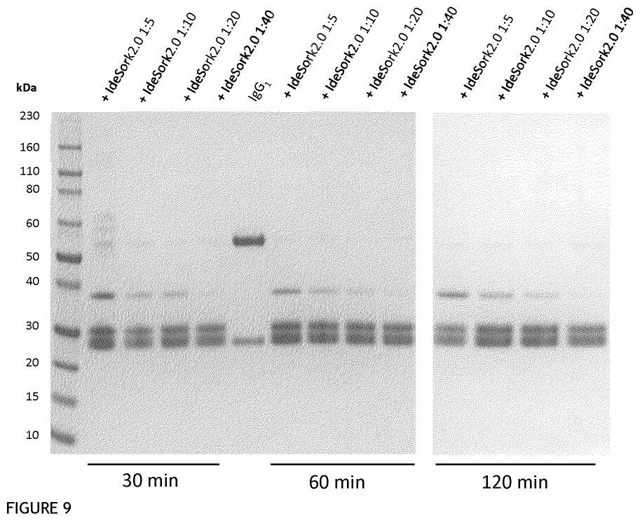

The optimal conditions for IdeSORK2.0 were assessed using a Box-Behnken experimental design to assess multiple factors and their inter-dependence. We assayed the importance of pH, NaCl and temperature for the protease, and the results are summarized in FIG. 7. IdeSORK2.0 is highly active in pH 7.0-8.5 and tolerates NaCl up to 200 mM without a significant drop in activity. The activity is improved when incubated at 37° C. as compared to room temperature. The enzymatic reaction is mainly driven by the substrate concentration (high concentration favours the reaction), while the enzyme concentration is secondary. This can also be seen as a low percentage IgG not being readily hydrolyzed even after a pro-longed incubation (FIG. 9). In summary: pH optimum (range): 7.5 (6.5-8.5; >90% activity), NaCl optimum (range): 0 mM (0-200 mM; >90% activity), no requirement for co-factors or reducing agents, high specificity for IgG.

### Time-Dose Assays

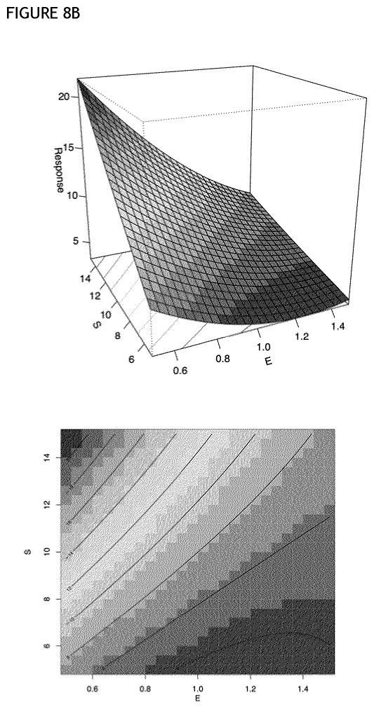

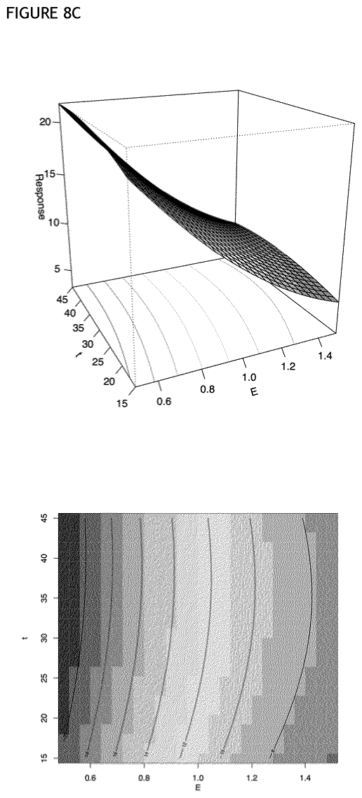

To investigate the impact of substrate and enzyme concentration, as well as time needed to complete (>95% hydrolysis) a reaction, a new Box-Behnken assay was designed (FIG. 8). The data was supported by traditional assays, with similar results (FIG. 9). IdeSORK2.0 quite rapidly hydrolyzes the vast majority of the material, but needs a longer time to finish hydrolysis of the last percentages of intact IgG, similar to other immunoglobulin proteases. A ratio of 1:40 hydrolyzes >95% of IgG in 30-60 minutes.

### Comparison to Other IgG Proteases

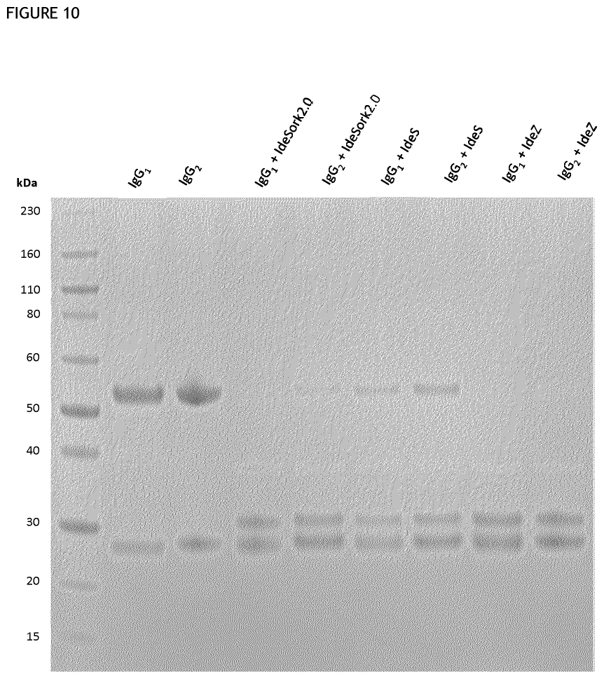

To further look into the activity of IdeSORK2.0, its activity was compared with IdeS and IdeZ against both IgG1 and IgG2. All enzymes were able to hydrolyze the antibodies in seemingly an identical fashion, though slightly different efficiently (FIG. 10).

### Sequence Comparison to Other IgG Proteases

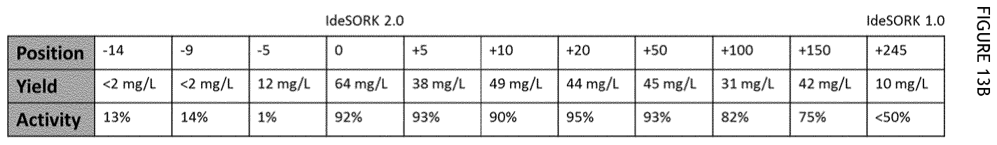

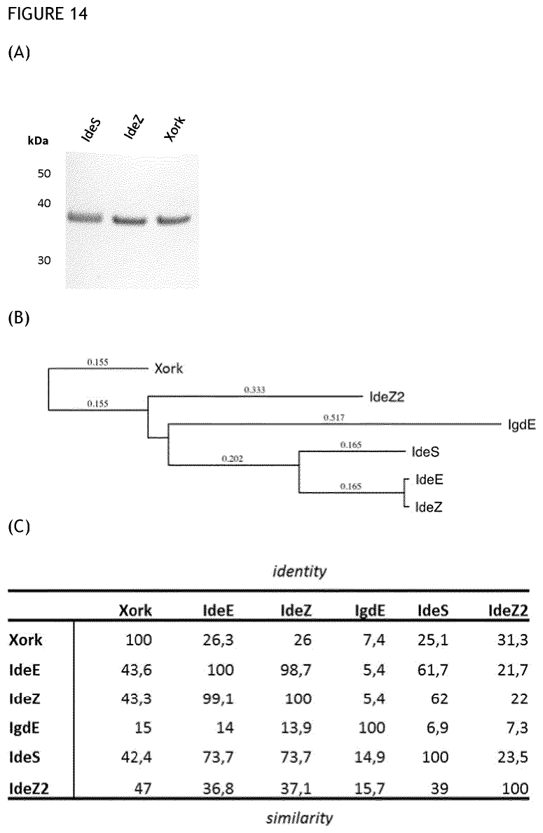

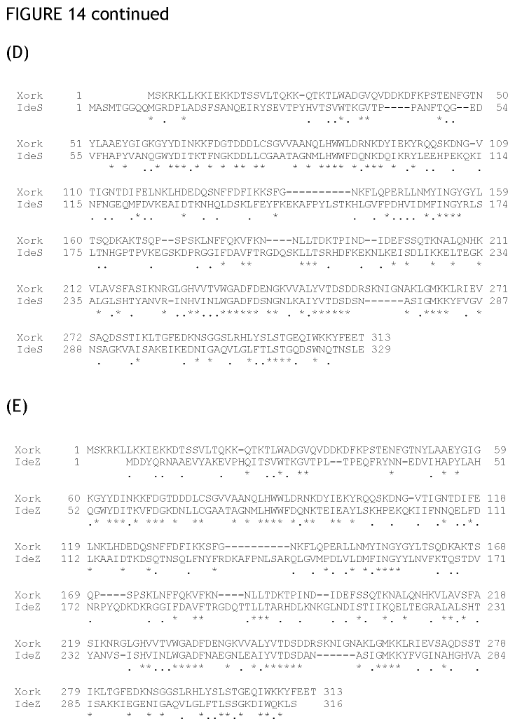

Clustal W alignment was performed for the sequences of IdeSORK, IdeSORK2.0, IdeS and IdeZ (FIG. 13A). Although comparable in length to IdeS, IdeSORK2.0 has only around 25% identity, 42% similarity to IdeS in the aligned sequence, which is significantly less than the similarity between IdeS and IdeZ. IdeS and IdeZ have 62% identity, 74% similarity with each other, and cluster together in a phylogenetic tree (FIG. 14B). Both IdeSORK and the truncated form IdeSORK2.0 are related to each other, but only distantly related to IdeS and IdeZ.

FIG. 14 shows the percentage similarity and identity of IdeSORK2.0 (Xork) to other known IgG hydrolases, as well as amino acid sequence alignments for Xork vs IdeS and IdeZ.

### Activity of IgG Hydrolases in Human Serum

Similar to IdeS and IdeZ, IdeSORK2.0 demonstrated immunoglobulin cleavage activity in the presence of human serum (FIG. 15). This demonstrates that IdeSORK2.0 has application in a clinical setting.

### Prevalence of Anti-IgG Hydrolase Antibodies in IVIG

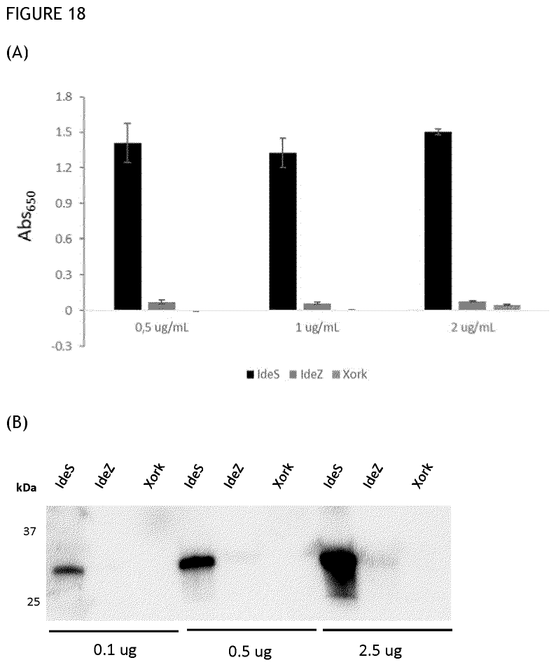

The prevalence of anti-IgG hydrolase antibodies in the human population was assessed by ELISA and Western blot analysis of IVIG binding to IdeS, IdeZ and IdeSORK2.0 was assessed (FIGS. 18A and B). Antibodies against IdeS were prevalent. There was significantly less antibody binding to IdeZ and even less for IdeSORK2.0. IdesSORK2.0 demonstrated very little reactivity with IVIG indicating that anti-IdesSORK2.0 antibodies are not common in humans.

### Materials & Methods

### Expression & Purification of Recombinant Protein

The vector (20 ng) was transformed into chemically competent E. coli BL21 DE3 STAR cells according to manufacturer's instructions (Invitrogen). Vector sequences are provided as SEQ ID NO: 18 for IdeSORK1.0 and SEQ ID NO: 19 for IdeSORK2.0. The vector used was pet21a+. An overnight culture of bacteria grown in LB supplemented with 100 μg/mL ampicillin, at 37° C., 200 rpm is used to inoculate fresh LB (supplemented with 100 μg/mL ampicillin) at a ratio of 1:40. The incubation continues until OD600 reaches ca 0.6-0.8 upon which the temperature is reduced to 30° C. and protein expression is induced by addition of 1 mM IPTG. Expression continues for 5 hours until the cells are collected and stored at −20° C. until further processing.

Frozen cells were thawed, and resuspended in binding buffer (5 mL/g cells; 20 mM sodium phosphate pH 7.4, 500 mM NaCl, 20 mM imidazole) before lysis through sonication. Insoluble cell debris was removed by centrifugation (11000 g, 20 min, 4° C.), and the lysate purified using a pre-equilibrated His GraviTrap before being buffer exchanged to TBS on a PD10 column. All material was analyzed on SDS-PAGE and Nanodrop for determination of purity and quality of the product.

### Specificity Determination Using LC-MS

Subunit fragments were denatured and reduced followed by reversed phase LC-MS analysis. Separation was achieved using a BioResolve RP mAb Polyphenol, 450 Å, 2.7 μm 2.1×100 mm, Waters (0.1% formic acid in water and 0.1% formic acid in 95% acetonitrile as mobile phases) and run on an Agilent 1260/1290 UHPLC system coupled to a Bruker Impact II QTOF mass spectrometer. The obtained data was evaluated in Bruker Compass DataAnalysis software using the MaxEnt algorithm for deconvolution and SNAP algorithm for determination of the monoisotopic molecular weights.

### General Protease Activity Determination

General protease activity was determined using the EnzChek Protease Assay kit, according to manufacturer's instructions. As a positive control the general protease FabULOUS (SpeB, 5 μg) was used, and as a negative control the IgG-specific protease FabRICATOR (IdeS, 10 μg). IdeSORK2.0 was added to a total quantity of 10 μg.

### Protease Inhibitor Assay

IgG1 (trastuzumab) was pre-incubated with a set of protease inhibitors (G-Biosciences) before addition of IdeSork2.0 (of SEQ ID NO: 3) (1:5), and the mixture was allowed to incubate overnight at 37° C. before analysis on SDS-PAGE.

### Immunoglobulin Specificity

Various antibodies and antibody based therapeutics were incubated with Xork, IdeS, or IdeZ (1:40 ratio) for 1 hour at 37° C. in PBS, with substrate concentrations ranging between 2-5 mg/mL, before being analyzed on SDS-PAGE. For kinetic studies, a substrate level of 2-200 μM IVIG was used during 10 min at 37° C. before being analyzed on a UHPLC system.

IgG that had been urea-denatured and reduced was incubated with IgG-specific enzymes at an E: S ratio of 1:10, incubating for 5 hours at 37° C. before analysis on SDS-PAGE.

For serum activity assays, an E: S ratio of 1:40 (assuming 10 mg/mL IgG in serum) was used, incubating 0-4 hours at 37° C. before analysis on a non-reduced SDS-PAGE. All enzymes were supplied by Genovis AB.

### Activity Assays

To better understand the optimal conditions needed for the enzyme to exert its activity, a Box-Behnken design was employed for the experiment. Addition of NaCl (0-400 mM), pH (5.5-8.5), [enzyme], [substrate], time, and temperature were parameters investigated. Unless otherwise noted, all experiments were conducted in TBS at an enzyme: substrate ratio of 1:20, for 1 hour at 37° C.

### ELISA

Proteins (IdeS, IdeZ, Xork; 0.5-2 μg/mL) were denatured by heat (70° C., 10 min) in 50 mM NaHCO3 pH 9.6. The enzymes were added to a 96 well plate (Maxisorb, NUNC) in triplicates and incubated o/n at 4° C. The wells were blocked for 2 h at RT in TBST skim milk (5%), washed, and a primary antibody added (IVIG; 0.005 mg/mL) diluted in TBST skim milk (1 h, 37° C.). The plate was washed before a secondary antibody (anti-human IgG-HRP, 1:3000) was added, continuing incubation for 1 h at 37° C. The wells were washed before 100 μl of TMB single solution was added. The plate incubated at RT in a plate reader with continuous reads for 45 minutes, measuring absorbance at 650 nm.

### Western Blot

IdeS, IdeZ and Xork (0.1-2.5 μg) were separated on an SDS-PAGE and transferred to a membrane. After o/n blocking at 4° C. towards TBST skim milk (5%) the primary antibody (IVIG, 5 μg/mL) was added and incubated for 2 h at RT. The membrane was washed, and a secondary antibody (anti-human IgG-HRP, 1:1000) was added. The membrane was thoroughly washed before addition of SuperSignal West Pico Plus chemiluminescent substrate was added. Signal was measured in a ChemiDoc.

### Sequences

SEQ ID NO: 1 (IdeSORK). Full length sequence of protease
isolated from Streptococcus krösus.
LSKRKLLKKIEKKDTSSVLTQKKQTKTLWADGVQVDDKDFKPSTENFGTNYLAAEYGI
GKGYYDINKKFDGTDDDLCSGVVAANQLHWWLDRNKDYIEKYRQQSKDNGVTTGNT
DIFELNKLHDEDQSNFFDFIKKSFGNKFLQPERLLNMYINGYGYLTSQDKAKTSQPSP
SKLNFFQKVFKNNLLTDKTPINDIDEFSSQTKNALQNHKVLAVSFASIKNRGLGHVVTV
WGADFDENGKVVALYVIDSDDRSKNTGNAKLGMKKLRIEVSAQDSSTIKLTGFEDKN
SGGSLRHLYSLSTGEQIWKKYFEETEKERIRLEEEADKAKLEQDRIQKEAEEKLALEKA
EKERIRLEEEADKAKLEQDRIQKEAEEKLALEKAEKERIRLEEEADKAKLEQDRIQKEAEE
KLALEKAEKERIRLEEEADKAKLEQDRIQKEAEEKLALEKAEKERIRLEEEAAKAKLEQEK
QIATAPQPDKKQENTTSEQEKPAPTELPPLVNKADETETPRETAPDQTPSATNTFRKIL
PKMNAVSQFFSQLMGTIQIVFAFILKIFK
SEQ ID NO: 2. N terminal fragment of IdeSORK having
immunoglobulin protease activity.
SKRKLLKKIEKKDTSSVLTQKKQTKTLWADGVQVDDKDFKPSTENFGTNYLAAEYGI
GKGYYDINKKFDGTDDDLCSGVVAANQLHWWLDRNKDYIEKYRQQSKDNGVTTGNT
DIFELNKLHDEDQSNFFDFIKKSFGNKFLQPERLLNMYINGYGYLTSQDKAKTSQPSP
SKLNFFQKVFKNNLLTDKTPINDIDEFSSQTKNALQNHKVLAVSFASIKNRGLGHVVTV
WGADFDENGKVVALYVTDSDDRSKNTGNAKLGMKKLRIEVSAQDSSTIKLTGFEDKN
SGGSLRHLYSLSTGEQIWKKYFEET
SEQ ID NO: 3 (IdeSORK2.0). Protein of SEQ ID NO: 2 engineered
to include an additional N terminal methionine and a
C-terminal protein purification tag - His6.
MSKRKLLKKIEKKDTSSVLTQKKQTKTLWADGVQVDDKDFKPSTENFGTNYLAAEYGI
GKGYYDINKKFDGTDDDLCSGVVAANQLHWWLDRNKDYIEKYRQQSKDNGVTTGNT
DIFELNKLHDEDQSNFFDFIKKSFGNKFLQPERLLNMYINGYGYLTSQDKAKTSQPSP
SKLNFFQKVFKNNLLTDKTPINDIDEFSSQTKNALQNHKVLAVSFASIKNRGLGHVVTV
WGADFDENGKVVALYVIDSDDRSKNTGNAKLGMKKLRIEVSAQDSSTIKLTGFEDKN
SGGSLRHLYSLSTGEQIWKKYFEETHHHHHH

SEQ ID NOS: 4 TO 10 ARE THE SEQUENCES OF THE HINGE/CH2 REGIONS
OF VARIOUS HUMAN AND MOUSE TGG SUBCLASSES

| Subclass | Hinge/CH2 | SEQ | Subclass | Hinge/CH2 | SEQ |
|---|---|---|---|---|---|
| Human | sequence | ID | Mouse | sequence | ID |
| IgG1 | CPPCPAPELLGGPSVF | 4 | IgG1 | PCICTVPEVSSVF | 10 |
| IgG2 | CPPCPAPPVAGPSVF | 5 | IgG2a | CPPCAAPNLLGGPSVF | 8 |
| IgG3 | CPRCPAPELLGGPSVF | 6 | IgG2b | CHKCPAPNLEGGPSVF | 11 |
| IgG4 | AHHAQAPEFLGGPSVF | 7 | IgG3 | GSSCPAGNILGGPSVF | 9 |

SEQ ID NO: 12. Full length IdeS. Also disclosed as NCBI Reference sequence
no. WP_010922160.1.
MRKRCYSTSAAVLAAVTLFVLSVDRGVIADSFSANQEIRYSEVTPYHVTSVWTKGVTPPANFTQGEDVFHAPYVA
NQGWYDITKTFNGKDDLLCGAATAGNMLHWWFDQNKDQIKRYLEEHPEKQKINFNGEQMEDVKEAIDTKNHQLDS
KLFEYFKEKAFPYLSTKHLGVFPDHVIDMFINGYRLSLINHGPTPVKEGSKDPRGGIFDAVETRGDQSKLLTSRH
DFKEKNLKEISDLIKKELTEGKALGLSHTYANVRINHVINLWGADFDSNGNLKAIYVTDSDSNASTGMKKYFVGV
NSAGKVAISAKEIKEDNTGAQVLGLFTLSTGQDSWNQIN
SEQ ID NO: 13. Mature IdeS. Also disclosed as Genbank accession no.
ADF13949.1.
DSFSANQEIRYSEVTPYHVTSVWTKGVTPPANFTQGEDVFHAPYVANQGWYDITKTENGKDDLLCGAATA
GNMLHWWFDQNKDQIKRYLEEHPEKQKINFNGEQMFDVKEAIDTKNHQLDSKLFEYFKEKAFPYLSTKHL
GVFPDHVIDMFINGYRLSLINHGPTPVKEGSKDPRGGIFDAVFTRGDQSKLLTSRHDFKEKNLKEISDLI
KKELTEGKALGLSHTYANVRINHVINLWGADFDSNGNLKAIYVTDSDSNASTGMKKYFVGVNSAGKVAIS
AKEIKEDNTGAQVLGLFTLSTGQDSWNQTN
SEQ ID NO: 14. Full length IdeZ. Also disclosed as NCBI Reference sequence
no. WP_014622780.1.
MKTIAYPNKPHSLSAGLLTAIAIFSLASSNITYADDYQRNATEAYAKEVPHQITSVWTKGVTPLTPEQFRYNNED
VIHAPYLAHQGWYDITKAFDGKDNLLCGAATAGNMLHWWFDQNKTEIEAYLSKHPEKQKIIFNNQELFDLKAAID
TKDSQTNSQLFNYFRDKAFPNLSARQLGVMPDLVLDMFINGYYLNVFKTQSTDVNRPYQDKDKRGGIFDAVETRG
DCTTLLTARHDLKNKGLNDISTIIKQELTEGRALALSHTYANVSISHVINLWGADENAEGNLEAIYVTDSDANAS
TGMKKYFVGINAHGHVAISAKKIEGENTGAQVLGLFTLSSGKDIWQKLS
SEQ ID NO: 15. Mature IdeZ.
DDYQRNATEAYAKEVPHQITSVWTKGVTPLTPEQFRYNNEDVIHAPYLAHQGWYDITKAFDGKDNLLCGAATAGN
MLHWWFDQNKTEIEAYLSKHPEKQKIIFNNQELFDLKAAIDTKDSQTNSQLFNYERDKAFPNLSARQLGVMPDLV
LDMFINGYYLNVFKTQSTDVNRPYQDKDKRGGIFDAVFTRGDQTTLLTARHDLKNKGLNDISTIIKQELTEGRAL
ALSHTYANVSISHVINLWGADFNAEGNLEAIYVTDSDANASTGMKKYFVGINAHGHVAISAKKIEGENTGAQVLG
LFTLSSGKDIWQKLS
SEQ ID NO: 16 is an exemplary nucleotide sequence encoding the polypeptide
of SEQ ID NO: 1 engineered for expression with an N terminal histidine and
a C terminal His6.
ATGAGCAAGCGTAAACTGCTGAAGAAAATCGAGAAGAAAGACACCAGCAGCGTGCTGACCCAGAAGAAACAAACCAAGACCCTGTGGGCGGATG
GCGTGCAGGTTGACGATAAGGACTTCAAACCGAGCACCGAAAACTTTGGTACCAACTACCTGGGGGGGGAGTATGGTATCGGCAAGGGTTACTA
CGATATCAACAAGAAGTTCGACGGCACCGACGATGACCTGTGCAGCGGTGTGGTTGCGGCGAACCAGCTGCACTGGTGGCTGGATCGTAACAAG
GACTACATTGAAAAATATCGTCAGCAAAGCAAGGATAACGGCGTGACCATCGGTAACACCGACATTTTCGAACTGAACAAACTGCACGATGAGG
ACCAGAGCAACTTCTTTGATTTCATCAAGAAAAGCTTCGGCAACAAGTTTCTGCAACCGGAGCGTCTGCTGAACATGTACATTAACGGCTACGG
TTATCTGACCAGCCAGGATAAGGCGAAAACCAGCCAACCGAGCCCGAGCAAGCTGAACTTCTTTCAGAAGGTTTTCAAAAACAACCTGCTGACC
GACAAAACCCCGATCAACGATATTGACGAATTTAGCAGCCAGACCAAGAACGCGCTGCAAAACCACAAAGTGCTGGCGGTTAGCTTCGCGAGCA
TCAAGAACCGTGGCCTGGGTCACGTGGTTACCGTGTGGGGCGCGGATTTTGACGAGAACGGTAAAGTGGTTGCGCTGTATGTTACCGACAGCGA
TGACCGTAGCAAGAACATTGGCAACGCGAAACTGGGTATGAAGAAACTGCGTATCGAAGTGAGCGCGCAGGATAGCAGCACCATTAAGCTGACC
GGCTTCGAGGACAAAAACAGCGGTGGCAGCCTGCGTCACCTGTACAGCCTGAGCACCGGTGAACAAATCTGGAAGAAGTACTTCGAGGAAACCG
AAAAGGAGCGTATTCGTCTGGAGGAAGAGGCGGATAAGGCGAAACTGGAACAGGACCGTATCCAAAAAGAGGCGGAAGAGAAACTGGCGCTGGA
AAAGGCGGAAAAAGAGCGTATCCGTCTGGAAGAGGAAGCGGACAAAGCGAAGCTGGAACAAGACCGTATTCAAAAAGAGGCGGAGGAAAAACTG
GCGCTGGAGAAAGCGGAGAAGGAACGTATCCGCCTGGAGGAAGAGGCGGACAAAGCGAAACTGGAACAAGATCGCATTCAGAAAGAGGCGGAGG
AGAAGCTGGCGCTGGAAAAAGCGGAGAAGGAGCGCATTCGCCTGGAAGAGGAAGCGGATAAAGCGAAGCTGGAACAGGATCGCATCCAGAAAGA
GGCGGAAGAGAAGCTGGCGCTGGAGAAGGCGGAGAAGGAGCGTATCCGACTGGAGGAAGAGGCGGCGAAAGCTAAACTGGAACAAGAAAAACAA
ATTGCGACCGCGCCGCAGCCGGACAAGAAACAAGAAAACACCACCAGCGAACAGGAGAAACCGGCGCCGACCGAGCTGCCGCCGCTGGTTAACA
AAGCGGATGAAACCGAGACCCCGCGTGAGACCGCGCCGGACCAAACCCCGAGCGCGACCAACACCTTCCGTAAGATCCTGCCGAAAATGAACGC
TGTGAGCCAGTTCTTTAGCCAACTGATGGGTACCATCCAGATTGTTTTCGCGTTTATCCTGAAGATTTTTAAAGGCAGCGGTCATCACCACCAC
SEQ ID NO: 17 is an exemplary nucleotide sequence encoding the polypeptide
of SEQ ID NO: 2 engineered for expression with an N terminal histidine and
a C terminal His₆. SEQ ID NO: 17 encodes SEQ ID NO: 3.
ATGAGCAAGCGTAAACTGCTGAAGAAAATCGAGAAGAAAGACACCAGCAGCGTGGTGACCCAGAAGAAACAAACCAAGACCCTGTGGGCGGATG
GCGTGCAGGTTGACGATAAGGACTTCAAACCGAGCACCGAAAACTTTGGTACCAACTACCTGGGGGGGGAGTATGGTATCGGCAAGGGTTACTA
CGATATCAACAAGAAGTTCGACGGCACCGACGATGACCTGTGCAGCGGTGTGGTTGCGGCGAACCAGCTCCACTGGTGGCTGGATCGTAACAAG
GACTACATTGAAAAATATCGTCAGCAAAGCAAGGATAACGGCGTGACCATCGGTAACACCGACATTTTCGAACTGAACAAACTGCACGATGAGG
ACCAGAGCAACTTCTTTGATTTCATCAAGAAAAGCTTCGGCAACAAGTTTCTGCAACCGGAGCGTCTGCTGAACATGTACATTAACGGCTACGG
TTATCTGACCAGCCAGGATAAGGCGAAAACCAGCCAACCGAGCCCGAGCAAGCTGAACTTCTTTCAGAAGGTTTTCAAAAACAACCTGCTGACC
GACAAAACCCCGATCAACGATATTGACGAATTTAGCAGCCAGACCAAGAACGCGCTGCAAAACCACAAAGTGCTGGCGGTTAGCTTCGCGAGCA
TCAAGAACCGTGGCCTGGGTCACGTGGTTACCGTGTGGGGCGCGGATTTTGACGAGAACGGTAAAGTGGTTGCGCTGTATGTTACCGACAGCGA
TGACCGTAGCAAGAACATTGGCAACGCGAAACTGGGTATGAAGAAACTGCGTATCGAAGTGAGCGCGCAGGATAGCAGCACCATTAAGCTGACC
GGCTTCGAGGACAAAAACAGCGGTGGCAGCCTGCGTCACCTGTACACCTGCATCACCACCACCACCACTAA
SEQ ID NO: 18 is an exemplary expression vector sequence comprising the
sequence of SEQ ID NO : 16. The vector is pET21a+. Gene is underlined.
TGGCGAATGGGACGCGCCCTGTAGCGGCGCATTAAGCGCGGCGGGTGTGGTGGTTACGCGCAGCGTGACCGCTACACTTGCCAGCGCCCTAGCG
CCCGCTCCTTTCGCTTTCTTCCCTTCCTTTCTCGCCACGTTCGCCGGCTTTCCCCGTCAAGCTCTAAATCGGGGGCTCCCTTTAGGGTTCCGAT
TTAGTGCTTTACGGCACCTCGACCCCAAAAAACTTGATTAGGGTGATGGTTCACGTAGTGGGCCATCGCCCTGATAGACGGTTTTTCGCCCTTT
GACGTTGGAGTCCACGTTCTTTAATAGTGGACTCTTGTTCCAAACTGGAACAACACTCAACCCTATCTCGGTCTATTCTTTTGATTTATAAGGG
ATTTTGCCGATTTCGGCCTATTGGTTAAAAAATGAGCTGATTTAACAAAAATTTAACGCGAATTTTAACAAAATATTAACGTTTACAATTTCAG
GTGGCACTTTTCGGGGAAATGTGCGCGGAACCCCTATTTGTTTATTTTTCTAAATACATTCAAATATGTATCCGCTCATGAGACAATAACCCTG
ATAAATGCTTCAATAATATTGAAAAAGGAAGAGTATGAGTATTCAACATTTCCGTGTCGCCCTTATTCCCTTTTTTGCGGCATTTTGCCTTCCT
GTTTTTGCTCACCCAGAAACGCTGGTGAAAGTAAAAGATGCTGAAGATCAGTTGGGTGCACGAGTGGGTTACATCGAACTGGATCTCAACAGCG
GTAAGATCCTTGAGAGTTTTCGCCCCGAAGAACGTTTTCCAATGATGATCACTTTTAAAGTTCTGCTATGTGGCGCGGTATTATCCCGTATTGA
CGCCGGGCAAGAGCAACTCGGTCGCCGCATACACTATTCTCAGAATGACTTGGTTGAGTACTCACCAGTCACAGAAAAGCATCTTACGGATGGC
ATGACAGTAAGAGAATTATGCAGTGCTGCCATAACCATGAGTGATAACACTGCGGCCAACTTACTTCTGACAACGATCGGAGGACCGAAGGAGC
TAACCGCTTTTTTGCACAACATGGGGGATCATGTAACTCGCCTTGATCGTTGGGAACCGGAGCTGAATGAAGCCATACCAAACGACGAGCGTGA
CACCACGATGCCTGCAGCAATGGCAACAACGTTGCGCAAACTATTAACTGGCGAACTACTTACTCTAGCTTCCCGGCAACAATTAATAGACTGG
ATGGAGGCGGATAAAGTTGCAGGACCACTTCTGCGCTCGGCCCTTCCGGCTGGCTGGTTTATTGCTGATAAATCTGGAGCCGGTGAGCGTGGGT
CTCGCGGTATCATTGCAGCACTGGGGCCAGATGGTAAGCCCTCCCGTATCGTAGTTATCTACACGACGGGGAGTCAGGCAACTATGGATGAACG
AAATAGACAGATCGCTGAGATAGGTGCCTCACTGATTAAGCATTGGTAACTGTCAGACCAAGTTTACTCATATATACTTTAGATTGATTTAAAA
CTTCATTTTTAATTTAAAAGGATCTAGGTGAAGATCCTTTTTGATAATCTCATGACCAAAATCCCTTAACGTGAGTTTTCGTTCCACTGAGCGT
CAGACCCCGTAGAAAAGATCAAAGGATCTTCTTGAGATCCTTTTTTTCTGCGCGTAATCTGCTGCTTGCAAACAAAAAAACCACCGCTACCAGC
GGTGGTTTGTTTGCCGGATCAAGAGCTACCAACTCTTTTTCCGAAGGTAACTGGCTTCAGCAGAGCGCAGATACCAAATACTGTCCTTCTAGTG
TAGCCGTAGTTAGGCCACCACTTCAAGAACTCTGTAGCACCGCCTACATACCTCGCTCTGCTAATCCTGTTACCAGTGGCTGCTGCCAGTGGCG
ATTAGTCGTGTCTTACCGGGTTGGACTCAAGACGATAGTTACCGGATAACGCGCAGCGGTCGGGCTGAACGGGGGGTTCGTGCACACAGCCCAG
CTTGGAGCGAACGACCTACACCGAACTGAGATACCTACAGCGTGAGCTATGAGAAAGCGCCACGCTTCCCGAAGGGAGAAAGGCGGACAGGTAT
CCGGTAAGCGGCAGGGTCGGAACAGGAGAGCGCACCAGGGAGCTTCCAGGGGGAAACGCCTGGTATCTTTATAGTCCTGTCGGGTTTCGCCACC
TCTGACGTTGAGCGTCGATTTTTGTGATGCTCGTCAGGGGGCGGAGCCTATGGAAAAACGCCAGCAACGCGGCCTTTTTACGGTTCCTGGCCTT
TTGCTGGCCTTTTGCTCACATGTTCTTTCCTGCGTTATCCCCTGATTCTGTGGATAACCGTATTACCGCCTTTGAGTGAGCTGATACCCCTCGC
CGCAGCCGAACGACCGAGCGCAGCGAGTCAGTGAGCGAGGAAGCGGAAGAGCGCCTGATGCGGTATTTTCTCCTTACGCATCTGTGCGGTATTT
CACACCGCATATATGGTGCACTCTCAGTACAATCTGCTCTGATGCCGCATAGTTAAGCCAGTATACACTCCGCTATCGCTACGTGACTGGGTCA
TGGCTGCGCCCCGACACCCGCCAACACCCGCTGACGCGCCCTGACGGGCTTGTCTGCTCCCGGCATCCGCTTACAGACAAGCTGTGACCGTCTC
CGGGAGCTGCATGTGTCAGAGGTTTTCACCGTCATCACCGAAACGCGCGAGGCAGCTGCGGTAAAGCTCATCAGCGTGGTCGTGAAGCGATTCA
CAGATGTCTGCCTGTTCATCCGCGTCCAGCTCGTTGAGTTTCTCCAGAAGCGTTAATGTCTGGCTTCTGATAAAGCGGGCCATGTTAAGGGCGG
TTTTTTCCTGTTTGGTCACTGATGCCTCCGTGTAAGGGGGATTTCTGTTCATGGGGGTAATGATACCGATGAAACGAGAGAGGATGCTCACGAT
ACGGGTTACTGATGATGAACATGCCCGGTTACTGGAACGTTGTGAGGGTAAACAACTGGCGGTATGGATGCGGCGGGACCAGAGAAAAATCACT
CAGGGTCAATGCCAGCGCTTCGTTAATACAGATGTAGGTGTTCCAGAGGGTAGCCAGCAGCATCCTGCGATGCAGATCCGGAACATAATGGTGC
AGGGCGCTGACTTCCGCGTTTCCAGACTTTACCAAACACGGAAACCGAAGACCATTCATGTTGTTGCTCAGGTCGCAGACGTTTTGCAGCAGCA
GTCGCTTCACCTTCGCTCGCGTATCGGTGATTCATTCTGCTAACCAGTAAGGCAACCCCGCCAGCCTAGCCGGGTCCTCAACGACAGGAGCACG
ATCATGCGCACCCGTGGGGCCGCCATGCCGGCGATAATGGCCTGCTTCTCGCCGAAACGTTTGGTGGCGGGACCAGTGACGAAGGCTTGAGCGA
GGGCGTGCAAGATTCCGAATACCGCAAGCGACAGGCCGATCATCGTCCCGCTCCAGCGAAAGCGCTCCTCGCCGAAAATGACCCAGAGCGCTGC
CGGCACCTGTCCTACGAGTTCCATGATAAAGAAGACACTCATAAGTGCGGCGACGATAGTCATCCCCCGCGCCCACCGGAAGGAGCTGACTGGG
TTGAAGGCTCTCAAGGGCATCGGTCGAGATCCCGGTGCCTAATGAGTGAGCTAACTTACATTAATTGCGTTGCGCTCACTGCCCGCTTTCCAGT
CGGGAAACCTGTCGTGCCAGCTGCATTAATGAATCGGCCAACGCGCGGGGAGAGGCGGTTTGCGTATTGGGCGCCAGGGTGGTTTTTCTTTTCA
CCAGTGAGACCGGCAACACCTGATTGCCCTTCACCCCCTGGCCCTGAGAGAGTTGCAGCAAGCGGTCCACGCTGGTTTGCCCCAGCAGGCGAAA
ATCCTGTTTGATGGTGGTTAACGGCGGGATATAACATGAGCTGTCTTCGGTATCGTCGTATCCCACTACCGAGATATCCGCACCAACGCGCAGC
CCGGACTCGSTAATGGCGCGCATTGCGCCCAGCGCCATCTGATCGTTGGCAACCAGCATCGCAGTGGGAACGATGCCCTCATTCAGCATTTGCA
TGGTTTGTTGAAAACCGGACATGGCACTCCAGTCGCCTTCCCGTTCCCCTATCGGCTGAATTTGATTGCGAGTGAGATATTTATCCCAGCCAGC
CAGACGCAGACGCGCCGAGACAGAACTTAATGGGCCCCCTAACAGCGCGATTTGCTGGTGACCCAATGCGACCAGATCCTCCACGCCCAGTCGC
GTACCGTCTTCATGGGAGAAMATAATACTGTTGATGGGTGTCTGGTCAGAGACATCAAGAAATAACGCCGGAACATTAGTGCAGGCAGCTTCCA
CAGCAATGGCATCCTGGTCATCCAGCGGATAGTTAATGATCAGCCCACTGACGCGTTGCGCGAGAAGATTGTGCACCGCCGCTTTACAGGCTTC
GACGCCGCTTCGTTCTACCATCGACACCACCACGCTGGCACCCAGTTGATCGGCGCGAGATTTAATCGCCGCGACAATTTGCGACGGCGCGTGC
AGGGCCAGACTGGAGGTCGCAACGCCAATCACCAACGACTCTTTGCCCCCCAGTTGTTGTGCCACGCGGTTCGGAATGTAATTCAGCTCCGCCA
TCGCCGCTTCCACTTTTTCCCGCGTTTTCGCAGAAACGTGGCTGGCCTGGTTCACCACGCGGGAAACGGTCTGATAAGAGACACCGGCATACTC
TGCGACATCGTATAACGTTACTGGTTTCACATTCACCACCCTGAATTGACTCTCTTCCGGGCGCTATCATGCCATACCGCGAAAGGTTTTGCGC
CATTCGATGGTGTCCGGGATCTCGACGCTCTCCCTTATGCGACTCCTGCATTAGGAACCACCCCAGTAGTACGTTGAGGCCGTTGAGCACCGCC
GCCGCAAGGAATGGTGCATCCAAGGAGATGGCGCCCAACAGTCCCCCGGCCACGGGGCCTGCCACCATACCCACGCCGAAACAAGCGCTCATGA
GCCCGAAGTGGCGAGCCCGATCTTCCCCATCGGTGATGTCGGCGATATAGGCGCCAGCAACCGCACCTGTGGCGCCGGTGATGCCGGCCACGAT
GCGTCCGGCGTAGAGGATCGAGATCTCGATCCCGCGAAATTAATACGACTCACTATAGGGGAATTGTGAGCGGATAACAATTCCCCTCTAGAAA
TAATTTTGTTTAACTTTAAGAAGGAGATATACATATGAGCAAGCGTAAACTGCTGAAGAAAATCGAGAAGAAAGACACCAGCAGCGTGCTGACC
CAGAAGAAACAAACCAAGACCCTGTGGGCGGATGGCGTCCAGGTTGACGATAAGGACTTCAAACCGAGCACCGAAAACTTTCGTACCAACTACC
TGGCGGCGGAGTATGGTATCGGCAAGGGTTACTACGATATCAACAAGAAGTTCGACGGCACCGACGATGACCTGTGCAGCGGTGTGGTTGCGGC
GAACCAGCTGCACTGGTGGCTGGATCGTAACAAGGACTACATTGAAAAATATCGTCACCAAAGCAAGGATAACGGCGTGACCATCGGTAACACC
GACATTTTCGAADTGAACAAACTGCACGATGAGGACCAGAGCAACTTCTTTGATTTCATCAAGAAAACCTTCGGCAACAAGTTTCTGCAACCGG
AGCGTCTGCTGAACATGTACATTAACGGCTACGGTTATCTGACCAGCCAGGATAAGGCGAAAACCAGCCAACCGAGCCCGAGCAAGCTGAACTT
CTTTCAGAAGGTTTTCAAAAACAACCTGCTGACCGACAAAACCCCGATCAACGATATTGACGAATTTAGCAGCCAGACCAAGAACGCGCTGCAA
AACCACAAAGTGCTGGCGGTTAGCTTCGCGAGCATCAAGAACCGTGGCCTGGGTCACGTGGTTACCGTGTGGGGCGCGGATTTTGACGAGAACG
GTAAAGTGGTTGCGCTGTATGTTACCGACAGCGATGACCGTAGCAAGAACATTCGCAACGCGAAACTGGGTATGAAGAAACTGCGTATCGAAGT
GAGCGCGCAGGATACCAGCACCATTAAGCTGACCCGCTTCGAGGACAAAAACAGCGGTGGCAGCCTGCGTCACCTGTACAGCCTGAGCACCCGT
GAACAAATCTGGAAGAAGTACTTCGAGGAAACCGAAAAGGAGCGTATTCGTCTGGAGGAAGAGGCGGATAAGGCGAAACTGGAACAGGACCGTA
TCCAAAAAGAGGCGGAAGAGAAACTGGCGCTGGAAAAGGCGGAAAAAGAGCGTATCCGTCTGGAAGAGGAAGCGGACAAAGCGAAGCTGGAACA
AGACCGTATTCAAAAAGAGGCGGAGGAAAAACTGGCGCTGGAGAAAGCGGAGAAGGAACGTATCCGCCTGGAGGAAGAGGCGGACAAAGCGAAA
CTGGAACAAGATCGCATTCAGAAAGAGGCGGAGGAGAAGCTGGCGCTGGAAAAAGCGGAGAAGGAGCGCATTCGCCTGGAAGAGGAAGCGGATA
AAGCGAAGCTGGAACAGGATCGCATCCAGAAAGAGGCGGAAGAGAAGCTGGCGCTGGAGAAGGCGGAGAAGGAGCGTATCCGACTGGAGGAAGA
GGCGGCGAAAGCTAAACTGGAACAAGAAAAACAAATTGCGACCGCGCCGCAGCCGGACAAGAAACAAGAAAACACCACCAGCGAACAGGAGAAA
CCGGCGCCGACCGAGCTGCCGCCGCTGGTTAACAAAGCGGATGAAACCGAGACCCCGCGTGAGACCGCGCCGGACCAAACCCCGAGCGCGACCA
ACAAATTCCGTAAGATCCTGCCGAAAATGAACGCTGTGAGCCAGTTCTTTAGCCAACTGATGGGTACCATCCAGATTGTTTTCGCGTTTATCCT
GAAGATTTTTAAAGGCAGCGGTCATCACCACCACCACCAGTAACTCGAGCACCACCACCACCACCACTGAGATGCGGCTGCTAACAAAGCCCGA
AAGGAAGCTGAGTTGGCTGCTGCCACCGCTGAGCAATAACTAGCATAACCCCTTGGGGCCTCTAAACGGGTCTTGAGGGGTTTTTTGCTGAAAG
GAGGAACTATATCCGGAT
SEQ ID NO: 19 is an exemplary expression vector sequence comprising the
sequence of SEQ ID NO : 17. The vector is pET21a+.
Gene is underlined.
TGGCGAATGGGACGCGCCCTGTAGCGGCGCATTAAGCGCGGCGGGTGTGGTGGTTACGCGCAGCGTGACCGCTACACTTGCCAGCGCCCTAGCG
CCCGCTCCTTTCGCTTTCTTCCCTTCCTTTCTCGCCACGTTCGCCGGCTTTCCCCGTCAAGCTCTAAATCGGGGGCTCCCTTTAGGGTTCCGAT
TTAGTGCTTTACGGCACCTCGACCCCAAAAAACTTGATTAGGGTGATCGTTCACGTAGTGGGCCATCGCCCTGATAGACGGTTTTTCCCCCTTT
GACGTTGGAGTCCACGTTCTTTAATAGTGGACTCTTGTTCCAAACTGGAACAACACTCAACCCTATCTCGGTCTATTCTTTTGATTTATAAGGG
ATTTTGCCGATTTCGGCCTTATTGGTTAAAAAATGAGCTGATTTAAGAAAATTTAACGCGAATTTTAACAAAATATTAACGTTTACAATTTCAG
GTGGCACTTTTCGGGGAAATGTGCGCGGAACCCCTATTTGTTTATTTTTCTAAATACATTCAAATATGTATCCGCTCATGAGACAATAACCCTG
ATAAATCCTTCAATAATATTGAAAAAGGAAGAGTATGAGTATTCAACATTTCCGTGTCGCCCTTATTCCCTTTTTTGCGGCATTTTGCCTTCCT
GTTTTTGCTCACCCAGAAACGCTGGTGAAACTAAAAGATGCTGAAGATCAGTTGGGTGCACGAGTGGGTTACATCGAACTGGATCTCAACAGCG
GTAAGATCCTTGAGAGTTTTCGCCCCGAAGAACGTTTTCCAATGATGAGCACTTTTAAAGTTCTGCTATGTGGCGCGGTATTATCCCGTATTGA
CGCCGGGCAAGAGCAACTCGGTCGCCGCATACACTATTCTCAGAATGACTTGGTTGAGTACTCACCAGTCACAGAAAAGCATCTTACGGATGGC
ATGACAGTAAGAGAATTATGCAGTGCTGCCATAACCATGAGTGATAACACTGCGGCCAACTTACTTCTGACAACGATCGGAGGACCGAAGGAGC
TAACCGCTTTTTTGGACAACATGGGGGATCATGTAACTCGCCTTGATCGTTGGGAACCGGAGCTGAATGAAGCCATACCAAACGACGAGCGTGA
CACCACGATGCCTGCAGCAATGGCAACAACGTTGCGCAAACTATTAACTGGCGAACTACTTACTCTAGCTTCCCGGCAACAATTAATAGACTGG
ATGGAGGCGGATAAAGTTGCAGGACCACTTCTGCGCTCGGCCCTTCCGGCTGGCTGGTTTATTGCTGATAAATCTGGAGCCGGTGAGGGTGGGT
CTCGGGGTATCATTGCAGCACTGGGGCCAGATGGTAAGCCCTCCCGTATCGTAGTTATCTACACGACGGGGAGTCAGGCAACTATGGATGAACG
AAATAGACAGATCGCTGAGATAGGTGCCTCACTGATTAAGCATTGGTAACTGTCAGACCAAGTTTACTCATATATACTTTAGATTGATTTAAAA
CTTCATTTTTAATTTAAAAGGATCTAGGTGAAGATCCTTTTTGATAATCTCATGACCAAAATCCCTTAACGTGAGTTTTCGTTCCACTGAGGGT
CAGACCCCGTAGAAAAGATCAAAGGATCTTCTTGAGATCCTTTTTTTCTGCGCGTAATCTGCTGCTTGCAAACAAAAAAACCACCGCTACCAGC
GGTGGTTTGTTTGCCGGATCAAGAGCTACCAACTCTTTTTCCGAAGGTAACTGGCTTCAGCAGAGCGCAGATACCAAATACTGTCCTTCTAGTG
TAGCCGTAGTTAGGCCACCACTTCAAGAACTCTGTAGCACCGCCTACATACCTCGCTCTGCTAATCCTGTTACCAGTGGCTGCTGCCAGTGGCG
ATAAGTCGTGTCTTACCGGGTTGGACTCAAGACGATAGTTACCGGATAAGGCGCAGCGGTCGGGCTGAACGGGGGGTTCGTGCACACAGCCCAG
CTTGGAGCGAACGACCTACACCGAACTGAGATACCTACAGCGTGAGCTATGAGAAAGCGCCACGCTTCCCGAAGGGAGAAAGGCGGACAGGTAT
CCGGTAAGCGGCAGGGTCGGAACAGGAGAGCGCACGAGGGAGCTTCCAGGGGGAAACGCCTGGTATCTTTATAGTCCTGTCGGGTTTCGCCACC
TCTGACTTGAGCGTCGATTTTTGTGATGCTCGTCAGGGGGGCGGAGCCTATGGAAAAACGCCAGCAACGCGGCCTTTTTACGGTTCCTGGCCTT
TTGCTGGCCTTTTGCTCACATGTTCTTTCCTGCGTTATCCCCTGATTCTGTGGATAACCGTATTACCGCCTTTGAGTGAGCTGATACCGCTCGC
CGCAGCCGAACGACCGAGCGCAGCGAGTCAGTGAGCGAGGAAGCGGAAGAGCGCCTGATGCGGTATTTTCTCCTTACGCATCTGTGCGGTATTT
CACACCGCATATATGGTGCACTCTCAGTACAATCTGCTCTGATGCCGCATAGTTAAGCCAGTATACACTCCGCTATCGCTACGTGACTGGGTCA
TGGCTGCGCCCCGACACCCGCCAACACCCGCTGACGCGCCCTGACGGGCTTGTCTGCTCCCGGCATCCGCTTACAGACAAGCTGTGACCGTCTC
CGGGAGCTGCATGTGTCAGAGGTTTTCACCGTCATCACCGAAACGCGCGAGGCAGCTGCGGTAAAGCTCATCAGCGTGGTCGTGAAGCGATTCA
CAGATGTCTGCCTGTTCATCCGCGTCCAGCTCGTTGAGTTTCTCCAGAAGCGTTAATGTCTGGCTTCTGATAAAGCGGGCCATGTTAAGGGCGG
TTTTTTCCTGTTTGGTCACTGATGCCTCCGTGTAAGGGGGATTTCTGTTCATGGGGGTAATGATACCGATGAAACGAGAGAGGATGCTCACGAT
ACGGGTTACTGATGATGAACATGCCCGGTTACTGGAACGTTGTGAGGGTAAACAACTGGCGGTATGGATGCGGCGGGACCAGAGAAAAATCACT
CAGGGTCAATGCCAGCGCTTCGTTAATACAGATGTAGGTGTTCCACAGGGTAGCCAGCAGCATCCTGCGATGCAGATCCGGAACATAATGGTGC
AGGGCGCTGACTTCCGCGTTTCCAGACTTTACGAAACACGGAAACCGAAGACCATTCATGTTGTTGCTCAGGTCGCAGACGTTTTGCAGCAGCA
GTCGCTTCACGTTCGCTCGCGTATCGGTGATTCATTCTGCTAACCAGTAAGGCAACCCCGCCAGCCTAGCCGGGTCCTCAACGACAGGAGCACG
ATCATGCGCACCCGTGGGGCCGCCATGCCGGCGATAATGGCCTGCTTCTCGCCGAAACGTTTGGTGGCGGGACCAGTGACGAAGGCTTGAGCGA
GGGCGTGCAAGATTCCGAATACCGCAAGCGACAGGCCGATCATCGTCGCGCTCCAGCGAAAGCGGTCCTCGCCGAAAATGACCCAGAGCGCTGC
CGGCACCTGTCCTACGAGTTGCATGATAAAGAAGACAGTCATAAGTGCGGCGACGATAGTCATGCCCCGCGCCCACCGGAAGGAGCTGACTGGG
TTGAAGGCTCTCAAGGGCATCGGTCGAGATCCCGGTGCCTAATGAGTGAGCTAACTTACATTAATTGCGTTGCGCTCACTGCCCGCTTTCCAGT
CGGGAAACCTGTCGTGGCAGCTGCATTAATGAATCGGCCAACGCGCGGGGAGAGGCGGTTTGCGTATTGGGCGCGAGGGTGGTTTTTCTTTTCA
CCAGTGAGACGGGCAACAGCTGATTGCCCTTCACCGCCTGGCCCTGAGAGAGTTGCAGCAAGCGGTCCACGCTGGTTTGCCCCAGCAGGCGAAA
ATCCTGTTTGATGGTGGTTAACGGCGGGATATAACATGAGCTGTCTTCGGTATCGTCGTATCCCACTACCGAGATATCCGCACCAACGCGCAGC
CCGGACTCGGTAATGGCGCGCATTGCGCCCAGCGCCATCTGATCGTTGGCAACCAGCATCGCAGTGGGAACGATGCCCTCATTCAGCATTTGCA
TGGTTTGTTGAAAACCGGACATGGCACTCCAGTCGCCTTCCCGTTCCGCTATCGGCTGAATTTGATTGCGAGTGAGATATTTATGCCAGCCAGC
CAGACGCAGACGCGCCGAGACAGAACTTAATGGGCCCGCTAACAGCGCGATTTGCTGGTGACCCAATGCGACCAGATGCTCCACGCCCAGTCGC
GTACCGTCTTCATGGGAGAAAATAATACTGTTGATGGGTGTCTGGTCAGAGACATCAAGAAATAACGCCGGAACATTAGTGCAGGCAGCTTCCA
CAGCAATGGCATCCTGGTCATCCAGCGGATAGTTAATGATCAGCCCACTGACGCGTTGCGCGAGAAGCTTGTGCACCGCCGCTTTACAGGCTTC
GACGCCGCTTCGTTCTACCATCGACACCACCACGCTGGCACCCAGTTGATCGGCGCGAGATTTAATCGCCGCGACAATTTGCGACGGCGCGTGC
AGGGCCAGACTGGAGGTGGCAACGCCAATCAGCAACGACTGTTTGCCCGCCAGTTGTTGTGCCACGCGGTTGGGAATGTAATTCAGCTCCGCCA
TCGCCGCTTCCACTTTTTCCCGCGTTTTCGCAGAAACGTGGCTGGCCTGGTTCACCACGCGGGAAACGGTCTGATAAGAGACACCGGCATACTC
TGCGACATCGTATAACGTTACTGGTTTCACATTCACCACCCTGAATTGACTCTCTTCCGGGCGCTATCATGCCATACCGCGAAAGGTTTTGCGC
CATTCGATGGTGTCCGGGATCTGGACGCTCTCCCTTATGCCACTCCTGCATTAGGAAGCAGCCCAGTAGTAGGTTGAGGCCGTTGAGCACCGCC
GCCGCAAGGAATGGTGCATGCAAGGAGATGGCGCCCAACAGTCCCCCGGCCACGGGGCCTGCCACCATACCCACGCCGAAACAAGCGCTCATGA
GCCCGAAGTGGCGAGCCCGATCTTCCCCATCGGTGATGTGGGCGATATAGGCGCCAGCAACCGCACCTGTGGCGCCGGTGATGCCGGCCACGAT
GCGTCCGGCGTAGAGGATCGAGATCTCGATCCCGCGAAATTAATACGACTCACTATAGGGGAATTGTGAGCGGATAACAATTCCCCTCTAGAAA
TAATTTTGTTTAACTTTAAGAAGGAGATATACATATGAGCAAGCGTAAACTGCTGAAGAAAATCGAGAAGAAAGACACCAGCAGCGTGCTGACC
CAGAAGAAACAAACCAAGACCCTGTGGGCGGATGGCGTGCAGGTTGACGATAAGGACTTCAAACCGAGCACCGAAAACTTTGGTACCAACTACC
TGGCGGCGGAGTATGGTATCGGCAAGGGTTACTACGATATCAACAAGAAGTTCGACGGCACCGACGATGACCTGTGCAGCGGTGTGGTTGCGGC
GAACCAGCTGCACTGGTGGCTGGATCGTAACAAGGACTACATTGAAAAATATCGTCAGCAAAGCAAGGATAACGGCGTGACCATCGGTAACACC
GACATTTTCGAACTGAACAAACTGCACGATGAGGACCAGAGCAACTTCTTTGATTTCATCAAGAAAAGCTTCGGCAACAAGTTTCTGCAACCGG
AGCGTCTGCTGAACATGTACATTAACGGCTACGGTTATCTGACCAGCCAGGATAAGGCGAAAACCAGCCAACCGAGCCCGAGCAAGCTGAACTT
CTTTCAGAAGGTTTTCAAAAACAACCTGCTGACCGACAAAACCCCGATCAACGATATTGACGAATTTAGCAGCCAGACCAAGAACGCGCTGCAA
AACCACAAAGTGCTGGCGGTTAGCTTCGCGAGCATCAAGAACCGTGGCCTGGGTCACGTGGTTACCGTGTGGGGCGCGGATTTTGACGAGAACG
GTAAAGTGGTTGCGCTGTATGTTACCGACAGCGATGACCGTAGCAAGAACATTGGCAACGCGAAACTGGGTATGAAGAAACTGCGTATCGAAGT
GAGCCGCGAGGATAGCAGCACCATTAAGCTGACCGGCTTCGAGGACAAAAACAGCGGTGGCAGCCTGCGTCACCTGTACAGCCTGAGCACCGGT
GAACAAATCTGGAAGAAGTACTTCGAGGAAACCCATCACCACCACCACCACTAACTCGAGCACCACCACCACCACCACTGAGATCCGGCTGCTA
ACAAAGCCCGAAAGGAAGCTGAGTTGGCTGCTGCCACCGCTGAGCAATAACTAGCATAACCCCTTGGGGCCTCTAAACGGGTCTTGAGGGGTTT
TTTGCTGAAAGGAGGAACTATATCCGGAT

1. A polypeptide, optionally an engineered polypeptide, having immunoglobulin protease activity, wherein said polypeptide comprises, consists essentially of, or consists of:
(a) the amino acid sequence of SEQ ID NO: 1;
(b) the amino acid sequence of SEQ ID NO: 2;
(c) an amino acid sequence which is an N terminal fragment of the sequence of SEQ ID NO: 1;
(d) an amino acid sequence which is at least 50% identical to the amino acid sequence of any one of (a), (b) or (c).

2. The polypeptide according to claim 1, is engineered to include an additional methionine at the N terminus and/or a protein purification or other tag at the C terminus, which tag may be joined to the C terminus by a linker.

3. The polypeptide according to claim 1, which comprises or consists of the amino acid sequence of SEQ ID NO: 3.

4. The polypeptide according to claim 1, which has protease activity against any immunoglobulin molecule comprising a CH2/hinge sequence as shown in any one of SEQ ID NOs: 4 to 8, wherein the polypeptide cleaves the said CH2/hinge sequence between the positions corresponding to positions 249 and 250 of human IgG according to the Kabat numbering system (positions 236 and 237 according to EU numbering system).

5. The polypeptide according to claim 1, which has protease activity against IgG, which may be human IgG1, IgG2, IgG3 or IgG4, mouse IgG2a or IgG3, or IgG from guinea pig or horse.

6. A polynucleotide or expression vector which comprises a nucleic acid sequence encoding a polypeptide of claim 1.

7. A host cell comprising the polynucleotide or expression vector of claim 6, which is preferably a bacterial cell, preferably not a cell of a streptococcal species, and most preferably a cell of E. coli.

8. The polypeptide according to claim 1, wherein the polypeptide is provided in solution, lyophilised, or immobilised, optionally together with an effective amount of at least one excipient, which is preferably a preservative.

9. A composition comprising a polypeptide according to claim 1 together with at least one excipient, which is preferably a preservative; optionally wherein the composition is pharmaceutically acceptable.

10. A method comprising administering a polypeptide according to claim 1, a polynucleotide or expression vector which comprises a nucleic acid sequence encoding said polypeptide, or a composition comprising said polypeptide together with at least one excipient, to a sample or a subject in which IgG is present.

11. An in vitro method comprising administering a polypeptide according to claim 1, a polynucleotide or expression vector which comprises a nucleic acid sequence encoding said polypeptide, or a composition comprising said polypeptide together with at least one excipient, to a sample in which IgG is present.

12. The method of claim 10, which is for the ex vivo cleavage of IgG in a sample, and which comprises administering said polypeptide to the sample and incubating under conditions suitable for IgG protease activity.

13. The method of claim 12 which additionally comprises the separation, detection or analysis of the resulting cleavage products, and/or wherein the method generates Fc and Fab fragments.

14.-15. (canceled)

16. The method of claim 10, which is for the prevention or treatment of a disease or condition in a subject, and which method comprises administering said polypeptide, polynucleotide, expression vector, or composition to the subject in a prophylactically or therapeutically effective amount.

17. The method of claim 16, wherein said disease or condition is a disease or condition mediated in whole or in part by pathogenic IgG antibodies.

18. The method of claim 16, wherein the disease or condition is (i) an autoimmune disease mediated in whole or in part by IgG antibodies; (ii) IgG mediated rejection of an organ or tissue transplant received by the subject; or (iii) IgG mediated anti-drug responses to a therapeutic agent administered to the subject.

19.-22. (canceled)

23. A method for improving the benefit to a subject of a therapeutic agent, such as a therapeutic antibody, comprising administering the polypeptide according to claim 1, the polynucleotide or expression vector which comprises a nucleic acid sequence encoding said polypeptide, or a composition comprising said polypeptide together with at least one excipient, which method comprises i) administering said polypeptide, polynucleotide, expression vector, or composition to the subject in a prophylactically or therapeutically effective amount and ii) administering, such as subsequently administering, a therapeutic agent, such as a therapeutic antibody to the subject.


SEQUENCE LISTING (27 sequences)

SEQ ID NO: 1 - protein, 557 aa, Artificial Sequence
  Full length sequence of protease isolated from Streptococcus krosus (IdeSORK)
LSKRKLLKKIEKKDTSSVLTQKKQTKTLWADGVQVDDKDFKPSTENFGTNYLAAEYGIGKGYYDINKKFDGTDDDLCSGVVAANQLHWWLDRNKDYIEKYRQQSKDNGVTIGNTDIFELNKLHDEDQSNFFDFIKKSFGNKFLQPERLLNMYINGYGYLTSQDKAKTSQPSPSKLNFFQKVFKNNLLTDKTPINDIDEFSSQTKNALQNHKVLAVSFASIKNRGLGHVVTVWGADFDENGKVVALYVTDSDDRSKNIGNAKLGMKKLRIEVSAQDSSTIKLTGFEDKNSGGSLRHLYSLSTGEQIWKKYFEETEKERIRLEEEADKAKLEQDRIQKEAEEKLALEKAEKERIRLEEEADKAKLEQDRIQKEAEEKLALEKAEKERIRLEEEADKAKLEQDRIQKEAEEKLALEKAEKERIRLEEEADKAKLEQDRIQKEAEEKLALEKAEKERIRLEEEAAKAKLEQEKQIATAPQPDKKQENTTSEQEKPAPTELPPLVNKADETETPRETAPDQTPSATNTFRKILPKMNAVSQFFSQLMGTIQIVFAFILKIFK

SEQ ID NO: 2 - protein, 312 aa, Artificial Sequence
  N terminal fragment of IdeSORK
SKRKLLKKIEKKDTSSVLTQKKQTKTLWADGVQVDDKDFKPSTENFGTNYLAAEYGIGKGYYDINKKFDGTDDDLCSGVVAANQLHWWLDRNKDYIEKYRQQSKDNGVTIGNTDIFELNKLHDEDQSNFFDFIKKSFGNKFLQPERLLNMYINGYGYLTSQDKAKTSQPSPSKLNFFQKVFKNNLLTDKTPINDIDEFSSQTKNALQNHKVLAVSFASIKNRGLGHVVTVWGADFDENGKVVALYVTDSDDRSKNIGNAKLGMKKLRIEVSAQDSSTIKLTGFEDKNSGGSLRHLYSLSTGEQIWKKYFEET

SEQ ID NO: 3 - protein, 319 aa, Artificial Sequence
  (IdeSORK2.0) Protein of SEQ ID NO 2 engineered to  include an additional N terminal methionine and a C-terminal protein purification tag - His6
MSKRKLLKKIEKKDTSSVLTQKKQTKTLWADGVQVDDKDFKPSTENFGTNYLAAEYGIGKGYYDINKKFDGTDDDLCSGVVAANQLHWWLDRNKDYIEKYRQQSKDNGVTIGNTDIFELNKLHDEDQSNFFDFIKKSFGNKFLQPERLLNMYINGYGYLTSQDKAKTSQPSPSKLNFFQKVFKNNLLTDKTPINDIDEFSSQTKNALQNHKVLAVSFASIKNRGLGHVVTVWGADFDENGKVVALYVTDSDDRSKNIGNAKLGMKKLRIEVSAQDSSTIKLTGFEDKNSGGSLRHLYSLSTGEQIWKKYFEETHHHHHH

SEQ ID NO: 4 - protein, 16 aa, Homo sapiens
CPPCPAPELLGGPSVF

SEQ ID NO: 5 - protein, 15 aa, Homo sapiens
CPPCPAPPVAGPSVF

SEQ ID NO: 6 - protein, 16 aa, Homo sapiens
CPRCPAPELLGGPSVF

SEQ ID NO: 7 - protein, 16 aa, Homo sapiens
AHHAQAPEFLGGPSVF

SEQ ID NO: 8 - protein, 16 aa, Mus musculus
CPPCAAPNLLGGPSVF

SEQ ID NO: 9 - protein, 16 aa, Mus musculus
GSSCPAGNILGGPSVF

SEQ ID NO: 10 - protein, 13 aa, Mus musculus
PCICTVPEVSSVF

SEQ ID NO: 11 - protein, 16 aa, Mus musculus
CHKCPAPNLEGGPSVF

SEQ ID NO: 12 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 13 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 14 - protein, 349 aa, Streptococcus equi
MKTIAYPNKPHSLSAGLLTAIAIFSLASSNITYADDYQRNATEAYAKEVPHQITSVWTKGVTPLTPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 15 - protein, 315 aa, Streptococcus equi
DDYQRNATEAYAKEVPHQITSVWTKGVTPLTPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 16 - DNA, 1701 bp, Artificial Sequence
  exemplary nucleotide sequence encoding the polypeptide of SEQ ID NO 1
atgagcaagcgtaaactgctgaagaaaatcgagaagaaagacaccagcagcgtgctgacccagaagaaacaaaccaagaccctgtgggcggatggcgtgcaggttgacgataaggacttcaaaccgagcaccgaaaactttggtaccaactacctggcggcggagtatggtatcggcaagggttactacgatatcaacaagaagttcgacggcaccgacgatgacctgtgcagcggtgtggttgcggcgaaccagctgcactggtggctggatcgtaacaaggactacattgaaaaatatcgtcagcaaagcaaggataacggcgtgaccatcggtaacaccgacattttcgaactgaacaaactgcacgatgaggaccagagcaacttctttgatttcatcaagaaaagcttcggcaacaagtttctgcaaccggagcgtctgctgaacatgtacattaacggctacggttatctgaccagccaggataaggcgaaaaccagccaaccgagcccgagcaagctgaacttctttcagaaggttttcaaaaacaacctgctgaccgacaaaaccccgatcaacgatattgacgaatttagcagccagaccaagaacgcgctgcaaaaccacaaagtgctggcggttagcttcgcgagcatcaagaaccgtggcctgggtcacgtggttaccgtgtggggcgcggattttgacgagaacggtaaagtggttgcgctgtatgttaccgacagcgatgaccgtagcaagaacattggcaacgcgaaactgggtatgaagaaactgcgtatcgaagtgagcgcgcaggatagcagcaccattaagctgaccggcttcgaggacaaaaacagcggtggcagcctgcgtcacctgtacagcctgagcaccggtgaacaaatctggaagaagtacttcgaggaaaccgaaaaggagcgtattcgtctggaggaagaggcggataaggcgaaactggaacaggaccgtatccaaaaagaggcggaagagaaactggcgctggaaaaggcggaaaaagagcgtatccgtctggaagaggaagcggacaaagcgaagctggaacaagaccgtattcaaaaagaggcggaggaaaaactggcgctggagaaagcggagaaggaacgtatccgcctggaggaagaggcggacaaagcgaaactggaacaagatcgcattcagaaagaggcggaggagaagctggcgctggaaaaagcggagaaggagcgcattcgcctggaagaggaagcggataaagcgaagctggaacaggatcgcatccagaaagaggcggaagagaagctggcgctggagaaggcggagaaggagcgtatccgactggaggaagaggcggcgaaagctaaactggaacaagaaaaacaaattgcgaccgcgccgcagccggacaagaaacaagaaaacaccaccagcgaacaggagaaaccggcgccgaccgagctgccgccgctggttaacaaagcggatgaaaccgagaccccgcgtgagaccgcgccggaccaaaccccgagcgcgaccaacaccttccgtaagatcctgccgaaaatgaacgctgtgagccagttctttagccaactgatgggtaccatccagattgttttcgcgtttatcctgaagatttttaaaggcagcggtcatcaccaccaccaccactaa

SEQ ID NO: 17 - DNA, 918 bp, Artificial Sequence
  exemplary nucleotide sequence encoding the polypeptide of SEQ ID NO 2
atgagcaagcgtaaactgctgaagaaaatcgagaagaaagacaccagcagcgtgctgacccagaagaaacaaaccaagaccctgtgggcggatggcgtgcaggttgacgataaggacttcaaaccgagcaccgaaaactttggtaccaactacctggcggcggagtatggtatcggcaagggttactacgatatcaacaagaagttcgacggcaccgacgatgacctgtgcagcggtgtggttgcggcgaaccagctgcactggtggctggatcgtaacaaggactacattgaaaaatatcgtcagcaaagcaaggataacggcgtgaccatcggtaacaccgacattttcgaactgaacaaactgcacgatgaggaccagagcaacttctttgatttcatcaagaaaagcttcggcaacaagtttctgcaaccggagcgtctgctgaacatgtacattaacggctacggttatctgaccagccaggataaggcgaaaaccagccaaccgagcccgagcaagctgaacttctttcagaaggttttcaaaaacaacctgctgaccgacaaaaccccgatcaacgatattgacgaatttagcagccagaccaagaacgcgctgcaaaaccacaaagtgctggcggttagcttcgcgagcatcaagaaccgtggcctgggtcacgtggttaccgtgtggggcgcggattttgacgagaacggtaaagtggttgcgctgtatgttaccgacagcgatgaccgtagcaagaacattggcaacgcgaaactgggtatgaagaaactgcgtatcgaagtgagcgcgcaggatagcagcaccattaagctgaccggcttcgaggacaaaaacagcggtggcagcctgcgtcacctgtacagcctgcatcaccaccaccaccactaa

SEQ ID NO: 18 - DNA, 7068 bp, Artificial Sequence
  exemplary expression vector sequence comprising the sequence of SEQ ID NO  16
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgagcaagcgtaaactgctgaagaaaatcgagaagaaagacaccagcagcgtgctgacccagaagaaacaaaccaagaccctgtgggcggatggcgtgcaggttgacgataaggacttcaaaccgagcaccgaaaactttggtaccaactacctggcggcggagtatggtatcggcaagggttactacgatatcaacaagaagttcgacggcaccgacgatgacctgtgcagcggtgtggttgcggcgaaccagctgcactggtggctggatcgtaacaaggactacattgaaaaatatcgtcagcaaagcaaggataacggcgtgaccatcggtaacaccgacattttcgaactgaacaaactgcacgatgaggaccagagcaacttctttgatttcatcaagaaaagcttcggcaacaagtttctgcaaccggagcgtctgctgaacatgtacattaacggctacggttatctgaccagccaggataaggcgaaaaccagccaaccgagcccgagcaagctgaacttctttcagaaggttttcaaaaacaacctgctgaccgacaaaaccccgatcaacgatattgacgaatttagcagccagaccaagaacgcgctgcaaaaccacaaagtgctggcggttagcttcgcgagcatcaagaaccgtggcctgggtcacgtggttaccgtgtggggcgcggattttgacgagaacggtaaagtggttgcgctgtatgttaccgacagcgatgaccgtagcaagaacattggcaacgcgaaactgggtatgaagaaactgcgtatcgaagtgagcgcgcaggatagcagcaccattaagctgaccggcttcgaggacaaaaacagcggtggcagcctgcgtcacctgtacagcctgagcaccggtgaacaaatctggaagaagtacttcgaggaaaccgaaaaggagcgtattcgtctggaggaagaggcggataaggcgaaactggaacaggaccgtatccaaaaagaggcggaagagaaactggcgctggaaaaggcggaaaaagagcgtatccgtctggaagaggaagcggacaaagcgaagctggaacaagaccgtattcaaaaagaggcggaggaaaaactggcgctggagaaagcggagaaggaacgtatccgcctggaggaagaggcggacaaagcgaaactggaacaagatcgcattcagaaagaggcggaggagaagctggcgctggaaaaagcggagaaggagcgcattcgcctggaagaggaagcggataaagcgaagctggaacaggatcgcatccagaaagaggcggaagagaagctggcgctggagaaggcggagaaggagcgtatccgactggaggaagaggcggcgaaagctaaactggaacaagaaaaacaaattgcgaccgcgccgcagccggacaagaaacaagaaaacaccaccagcgaacaggagaaaccggcgccgaccgagctgccgccgctggttaacaaagcggatgaaaccgagaccccgcgtgagaccgcgccggaccaaaccccgagcgcgaccaacaccttccgtaagatcctgccgaaaatgaacgctgtgagccagttctttagccaactgatgggtaccatccagattgttttcgcgtttatcctgaagatttttaaaggcagcggtcatcaccaccaccaccactaactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 19 - DNA, 6327 bp, Artificial Sequence
  exemplary expression vector sequence comprising the sequence of SEQ ID NO  17
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgagcaagcgtaaactgctgaagaaaatcgagaagaaagacaccagcagcgtgctgacccagaagaaacaaaccaagaccctgtgggcggatggcgtgcaggttgacgataaggacttcaaaccgagcaccgaaaactttggtaccaactacctggcggcggagtatggtatcggcaagggttactacgatatcaacaagaagttcgacggcaccgacgatgacctgtgcagcggtgtggttgcggcgaaccagctgcactggtggctggatcgtaacaaggactacattgaaaaatatcgtcagcaaagcaaggataacggcgtgaccatcggtaacaccgacattttcgaactgaacaaactgcacgatgaggaccagagcaacttctttgatttcatcaagaaaagcttcggcaacaagtttctgcaaccggagcgtctgctgaacatgtacattaacggctacggttatctgaccagccaggataaggcgaaaaccagccaaccgagcccgagcaagctgaacttctttcagaaggttttcaaaaacaacctgctgaccgacaaaaccccgatcaacgatattgacgaatttagcagccagaccaagaacgcgctgcaaaaccacaaagtgctggcggttagcttcgcgagcatcaagaaccgtggcctgggtcacgtggttaccgtgtggggcgcggattttgacgagaacggtaaagtggttgcgctgtatgttaccgacagcgatgaccgtagcaagaacattggcaacgcgaaactgggtatgaagaaactgcgtatcgaagtgagcgcgcaggatagcagcaccattaagctgaccggcttcgaggacaaaaacagcggtggcagcctgcgtcacctgtacagcctgagcaccggtgaacaaatctggaagaagtacttcgaggaaacccatcaccaccaccaccactaactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 20 - protein, 566 aa, Artificial Sequence
  IdeSork1.0
MSKRKLLKKIEKKDTSSVLTQKKQTKTLWADGVQVDDKDFKPSTENFGTNYLAAEYGIGKGYYDINKKFDGTDDDLCSGVVAANQLHWWLDRNKDYIEKYRQQSKDNGVTIGNTDIFELNKLHDEDQSNFFDFIKKSFGNKFLQPERLLNMYINGYGYLTSQDKAKTSQPSPSKLNFFQKVFKNNLLTDKTPINDIDEFSSQTKNALQNHKVLAVSFASIKNRGLGHVVTVWGADFDENGKVVALYVTDSDDRSKNIGNAKLGMKKLRIEVSAQDSSTIKLTGFEDKNSGGSLRHLYSLSTGEQIWKKYFEETEKERIRLEEEADKAKLEQDRIQKEAEEKLALEKAEKERIRLEEEADKAKLEQDRIQKEAEEKLALEKAEKERIRLEEEADKAKLEQDRIQKEAEEKLALEKAEKERIRLEEEADKAKLEQDRIQKEAEEKLALEKAEKERIRLEEEAAKAKLEQEKQIATAPQPDKKQENTTSEQEKPAPTELPPLVNKADETETPRETAPDQTPSATNTFRKILPKMNAVSQFFSQLMGTIQIVFAFILKIFKGSGHHHHHH

SEQ ID NO: 21 - protein, 335 aa, Artificial Sequence
  IdeS
MASMTGGQQMGRDPLADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNSLEHHHHHH

SEQ ID NO: 22 - protein, 322 aa, Artificial Sequence
  IdeZ
MDDYQRNATEAYAKEVPHQITSVWTKGVTPLTPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLSHHHHHH

SEQ ID NO: 23 - protein, 313 aa, Artificial Sequence
  Xork
MSKRKLLKKIEKKDTSSVLTQKKQTKTLWADGVQVDDKDFKPSTENFGTNYLAAEYGIGKGYYDINKKFDGTDDDLCSGVVAANQLHWWLDRNKDYIEKYRQQSKDNGVTIGNTDIFELNKLHDEDQSNFFDFIKKSFGNKFLQPERLLNMYINGYGYLTSQDKAKTSQPSPSKLNFFQKVFKNNLLTDKTPINDIDEFSSQTKNALQNHKVLAVSFASIKNRGLGHVVTVWGADFDENGKVVALYVTDSDDRSKNIGNAKLGMKKLRIEVSAQDSSTIKLTGFEDKNSGGSLRHLYSLSTGEQIWKKYFEET

SEQ ID NO: 24 - protein, 329 aa, Artificial Sequence
  IdeS
MASMTGGQQMGRDPLADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNSLE

SEQ ID NO: 25 - protein, 316 aa, Artificial Sequence
  IdeZ
MDDYQRNATEAYAKEVPHQITSVWTKGVTPLTPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 26 - protein, 22 aa, Artificial Sequence
  Digestion site
CPPCPAPELLGGPSVFLFPPKP

SEQ ID NO: 27 - protein, 22 aa, Artificial Sequence
  Digestion site
CPPCPAPEAAGGPSVFLFPPKP